(19) United States
(12) Patent Application Publication
(10) Pub. No.: US 2011/0301055 A1
(43) Pub. Date: Dec. 8, 2011

(54) METHODS FOR DETERMINING A PROGNOSIS IN MULTIPLE MYELOMA

(76) Inventors: Nicholas James Dickens, London (GB); Brian Andrew Walker, London (GB); Gareth John Morgan, London (GB)

(21) Appl. No.: 13/132,422

(22) PCT Filed: Dec. 4, 2009
(86) PCT No.: PCT/GB2009/002815

§ 371 Date: Aug. 25, 2011

Related U.S. Application Data
(60) Provisional application No. 61/120,178, filed on Dec. 5, 2008.

(51) Int. Cl. C40B 30/04; C12Q 1/68

(52) U.S. Cl. 506/9; 435/6.12

(57) ABSTRACT

Methods for determining a prognosis in multiple myeloma are disclosed, and in particular to methods that are capable of identifying patients with a poor prognosis and/or for determining the likelihood of a patient responding to a particular treatment. The methods identify myeloma samples having homozygous deletions in cell death genes, with dysregulated expression of 97 cell death genes forming a cell death expression signature, which is associated with poor prognosis in multiple myeloma. In a preferred aspect, three gene pairs, were found to provide a prognostic a “six gene signature” based on BUB1B and HDAC3; CDC2 and FIS1; and RAD21 and ITM2B (high expressors and low expressors respectively).

### FIELD OF THE INVENTION

The present invention relates to methods for determining a prognosis in multiple myeloma, and in particular to methods that are capable of identifying patients with a poor prognosis. These methods may be useful for determining the likelihood of a patient responding to a particular treatment and for helping to determine appropriate treatments for patients with multiple myeloma.

### BACKGROUND OF THE INVENTION

Multiple myeloma is the second most prevalent blood cancer (10%) after non-Hodgkin's lymphoma and represents approximately 1% of all cancers and 2% of all cancer deaths. Although the peak age of onset of multiple myeloma is 65 to 70 years of age, recent statistics indicate both increasing incidence and earlier age of onset. While multiple myeloma is regarded as incurable, there are a range of treatments, including chemotherapy and stem cell transplantation, that have been shown to significantly extend patient survival such that survival post diagnosis is presently about three years.

While there have been significant improvements in the treatment of myeloma, there remains a clinical need to identify high risk (HR) patients for whom alternate treatment strategies can be explored. The International Staging System (ISS) does this for groups of patients and is based on clinical factors which are surrogates for disease biology. Chromosomal analysis offers an alternative strategy and both cytogenetics and fluorescent in situ hybridisation (FISH) can be used to group myeloma into biologically relevant prognostically important groups.¹⁻⁴ It can be concluded that perhaps the most clinically important deletional event is loss of 17p and, while del(13) identified by cytogenetics has been suggested to have a poor prognosis, it is not so important when identified by FISH. Translocations into the IgH locus are associated with distinct disease subgroups and the t(4;14) has a poor prognosis and the t(11;14) a good prognosis, but within each of the groups there is variability in clinical outcome such that on their own they lack specificity in defining high risk cases.

It therefore remains a problem in the art to identify patient groups with multiple myeloma, in particular those patients with high risk myeloma and poor prognosis, so that treatment can be tailored to those groups.

### SUMMARY OF THE INVENTION

The present inventors have developed a new approach to identifying patient groups with multiple myeloma, by combining analysis of genetic change taking place during disease progression with global expression analysis. The inventors have shown the surprising importance of alterations in networks of gene expression in predicting clinical outcome in multiple myeloma. In particular, the inventors have shown the importance of alterations in expression of cell death genes.

Broadly, the present invention is based on the application of array based technologies to characterize regions of copy number variation relevant to the pathogenesis of myeloma. Based on the results of these initial studies, the present inventors investigated the full range of genes inactivated by the loss of genetic material occurring during disease progression as a means to identify genes and gene expression signatures with prognostic significance. Homozygous deletions (HDels) are particularly relevant in this respect, as by definition they contain genes that are inactivated on both alleles.

Accordingly, the present invention is based on experiments in which 500K high density arrays (2.5 kb resolution) were used to identify the location and frequency of HDels in presenting patient samples collected from a randomized clinical trial. In combination with global gene expression data, the number of target genes was filtered with the aim of identifying key pathologically relevant signatures and pathways.

The present inventors used homozygous deletions affecting genes in the Gene Ontology (GO) defined cell death genes to identify a patient group with poor prognosis, and used this to define a more generally applicable cell death expression signature (a 97 gene signature) which was further validated in two additional large data sets. Surprisingly, the 97 gene signature could be used to identify dysreguation of the cell death gene network (and thereby identify high risk patients) in samples without known homozygous deletions. Significantly, the present inventors unexpectedly found that it is possible to identify the same high risk cases with high specificity using only three pairs of genes from the 97 gene signature, thereby providing a readily applicable test for identifying high risk myeloma patients.

The present inventors found that in myeloma samples having HDels in cell death genes, the expression of 97 cell death genes is altered. Dysregulated expression of these 97 genes forms a cell death expression signature, which is associated with poor prognosis in multiple myeloma.

The present inventors found that the 97 genes clustered into two expression groups, a high expressor gene group and a low expressor gene group (Table 1). It was found that the relative expression levels of genes in a gene pair, wherein a gene pair consists of one high expressor gene and one low expressor gene, could be used to determine the prognosis of an individual having multiple myeloma. The surprising discovery that the relative expression levels of a limited subset of genes from the 97 gene signature can be used to sensitively predict poor prognosis in multiple myeloma provides a relatively quick, simple and sensitive way of assigning individuals with multiple myeloma patients to a high risk (poor prognosis) patient group.

Importantly, the present inventors discovered the prognostic significance of three gene pairs, forming a “six gene signature”. These three gene pairs were: BUB1B and HDAC3; CDC2 and FIS1; and RAD21 and ITM2B (high expressors and low expressors respectively). The present study showed that if the expression of the high expressor is greater than or equal to the expression of the low expressor (a high expression ratio) for any one of these three gene pairs in a sample from a myeloma patient, this is associated with a poor prognosis for that patient.

Accordingly, in a first aspect, the present invention provides a method for determining a prognosis for an individual having multiple myeloma, the method comprising:
determining the expression signature status of cell death genes in a sample obtained from the individual; and
using the expression signature status to determine a prognosis for the individual.

In particular, the present invention provides a method for determining a prognosis for an individual with multiple myeloma, the method comprising:
determining the expression signature status of cell death genes in a sample obtained from the individual, comprising;
determining the relative expression in the sample of each gene in one or more of the following gene pairs:
   a) the gene pair in which the first gene is BUB1B and the second gene is HDAC3,
   b) the gene pair in which the first gene is CDC2 and the second gene is FIS1,
   c) the gene pair in which the first gene is RAD21 and the second gene is ITM2B,
      and,
using the expression signature status to determine the prognosis for the individual.

The present invention further provides a method as described above wherein the expression signature status is used to determine the prognosis for the individual by assigning the individual to a high risk group if, for any one of the gene pairs, expression of the first gene is greater than or equal to expression of the second gene.

In a preferred embodiment, the present invention provides a method determining a prognosis for an individual having myeloma, wherein the individual is assigned to a high risk group if expression of BUB1B is greater than or equal to expression of HDAC3 in a sample obtained from the individual.

The invention further provides a method of determining a prognosis for an individual, wherein the individual is assigned to a high risk group if, in a sample obtained from the individual, expression of BUB1B is greater than or equal to expression of HDAC3, and the expression of a cell death gene belonging to a high expressor group is greater than or equal to expression of a cell death gene belonging to a low expressor group. Methods of identifying expressor groups and gene pairs useful in determining prognosis are described in detail below.

In a preferred embodiment, the invention also provides a method determining a prognosis for an individual, wherein the individual is assigned to a high risk group if expression of BUB1B is greater than or equal to expression of HDAC3, and expression of CDC2 is greater than or equal to expression of FIS1, and/or expression of RAD21 is greater than or equal to expression of ITM2B.

Methods of determining the expression signature status of cell death genes, and of determining the relative expression of genes in gene pairs are described in more detail below.

The present inventors found that myeloma samples having a homozygous deletion in particular, cell death genes were associated with poor prognosis.

Accordingly, in a further aspect, the present invention provides a method for determining a prognosis for an individual with multiple myeloma, the method comprising:
determining whether the patient has a homozygous deletion in any one of the following cell death genes:
FAF1, CDKN2C, CTSB, TNFRSF10B, TNFRSF10D, BIRC2, BIRC3, ESR1, PLAGL1, SGK, EMP1, FGF14, FOXO1, TFDP1, KRT18, and
wherein the presence of a homozygous deletion indicates a poor prognosis.

In a further aspect, the present invention provides a method for identifying an expression signature of cell death genes, the status of which signature is suitable for determining a prognosis for an individual having multiple myeloma, the method comprising:
a) obtaining tumour cell samples from a set of individuals having multiple myeloma,
b) identifying homozygous deletions in the samples,
c) determining which genes having homozygous deletions are cell death genes,
d) identifying samples having homozygous deletions in cell death genes, and determining which genes are differentially expressed in the identified samples relative to the samples which do not have homozygous deletions in cell death genes,
e) identifying which of the differentially expressed genes is itself a cell death gene,
f) performing hierarchical cluster analysis on the sample set, to determine whether differential expression of the genes identified in step (e) is associated with altered overall survival and/or progression-free survival of individuals in the set, and
g) assigning the genes identified in step (e) to a gene expression signature if their differential expression is associated with altered overall survival and or progression free survival in step.

The present inventors developed the above method and used it to identify the 97 gene signature.

In a further aspect, the invention provides a method of obtaining a refined expression signature for cell death genes, the status of which signature is suitable for determining a prognosis for an individual having multiple myeloma, the method comprising:
(a) identifying an expression signature of cell death genes according to the above described the method,
(b) performing hierarchical cluster analysis on the expression of genes in the expression signature to cluster the genes into an A group and a B group, each group having a distinct expression pattern
(c) identifying pairs of genes, each pair comprising a first gene from the A group and a second gene from the B group
(d) determining for each sample in a set of samples obtained from individuals having myeloma the relative expression of each gene in a plurality of gene pairs,
(e) classifying each gene pair for each sample as “high ratio” if expression of the gene from group A is greater than or equal to expression of the gene from group B in the sample, or “low ratio” if the expression of the gene from group A is less than expression of the gene from group A in the sample, and,
(f) performing statistical analysis to determine whether a gene pair having a high ratio or low ratio in a sample is associated with altered overall survival and/or progression free survival for the individual from which the sample was obtained.

The present inventors used the above method to cluster genes from the 97 gene signature into a group of high expressor genes and a group of low expressor genes. Gene pairs comprising one high expressor and one low expressor gene were generated and their relative expression determined in each sample of a set of samples obtained from myeloma patients. A high ratio of expression (i.e. expression ratio of high expressor versus low expressor gene greater than or equal to 1) for any one of three gene pairs was associated with poor prognosis (lower median OS and PFS). The three gene pairs identified in this way form the six gene signature.

The above described method may be performed in order to identify additional gene pairs for which a high ratio in a sample is associated with poor prognosis. In particular, the above described method may be used (possibly on other myeloma sample sets, preferably on larger myeloma sets) to refine the 97 gene signature to identify further gene pairs having prognostic value. These gene pairs may be used in combination with one or more gene pairs from the six gene signature in order to determine a prognosis for a myeloma patient.

The terms “high expressor”, “high expressor gene”, “high expressor cluster”, “high expressor cell death gene cluster” and “high expressor group” refer to those genes identified in Table 1 as belonging to the high expressor class. Similarly, the terms “low expressor, “low expressor gene”, “low expressor cluster”, “low expressor cell death gene cluster” and “gene from a low expressor group” refer to the genes identified in Table 1 as belonging to the low expressor class.

The term “cell death gene” when used herein refers to a gene which is identified as having a function in a cell death pathways. In particular the term “cell death gene” refers to a gene identified using Gene Ontology (GO) annotation as a gene involved in a cell death pathway. The GO term “cell death” includes autolysis and programmed dell death, programmed cell death is an umbrella term that includes both apoptotic and non-apoptotic programmed cell death.

Patients assigned to a high risk patient group have a poor prognosis. A patient having poor prognosis is expected to have a shorter overall survival. (OS) and/or a shorter progression fee survival (PFS) relative to a control group. In particular, patient groups having a poor prognosis have, or are expected to have, a lower median OS and/or PFS than a control group. In the present study, control groups comprised myeloma patients that did not have particular HDels and/or were not positive for the 97 gene signature or the 6 gene signature.

The terms “97 gene signature”, “97 gene list” and “97 genes” refer to the group of cell death genes identified by the present inventors has having altered expression in myeloma samples having homozygous deletions in cell death genes (see Table 1). The term “98 gene signature” may be used to refer to the 97 gene signature (the 97 gene signature includes the cell death genes CDKN2C and FAF1, which may be difficult to distinguish using the SNP mapping methods described herein and can therefore be considered as one entity).

The terms “six gene signature”, “six gene list” and “six genes” and “three gene pairs” all refer to the group of cell death genes consisting of the following three gene pairs: BUB1B and HDAC3; CDC2 and FIS1; and RAD21 and ITM2B. A sample in which the expression of the first gene is greater than expression of the second gene for any one of the three pairs is positive for the six gene signature. The present inventors found that individuals whose samples were positive for the six gene signature had a poor prognosis relative to individuals negative for the six gene signature.

The term “expression ratio” refers to the ratio of expression levels in a sample of a high expressor gene versus a low expressor gene in a particular gene pair. In particular, it refers to the ratio of the amount of high expressor gene mRNA versus low expressor gene mRNA in a sample. The expression ratio is used as indicator of the relative expression of high expressor and low expressor genes. In the methods of the present invention an expression ratio (R) of greater than or equal to 1 (R≧1) indicates that there is more high expressor mRNA than low expressor mRNA in a sample, this may be referred to as a “high ratio” for that gene pair, indicating a “positive” result for the sample tested.

Preferably, a high ratio is a greater than 1.0 (R>1), i.e. the ratio of expression of the high expressor gene versus the low expressor gene in a gene pair is more than 1.0. More preferably a high ratio is greater than 1.1, 1.2, 1.3, 1.4, 1.5, 1.6, 1.7, 1.8, 1.9, 2.0, 2.5, 3.0, or 5.0.

The present invention includes the combination of the aspects and preferred features described except where such a combination is clearly impermissible or is stated to be expressly avoided. Embodiments of the present invention will now be described by way of example and not limitation with reference to the accompanying figures.

### BRIEF DESCRIPTION OF THE FIGURES

FIG. 1. Schematic of the filtering process to identify homozygous deletions important in myeloma, along with the number of SNPs, regions, or genes identified at each step.

FIG. 2. Positions of homozygously deleted genes on the genome. Genes with HDel in at least 2 samples with loss of expression in all HDel samples and genes HDel in at least 5% of samples.

FIG. 3. Kaplan-Meier survival curves for samples with deletions of a Cell Death gene (lower curves, grey) against those without in Myeloma IX (upper curves, black). Top panel shows OS—overall Survival (days), bottom panel shows PFS—progression-free survival (days). Median OS with deletion 26 months, without 47 months. OS Log rank p=0.0153 (N_{cell death deletion}=24, N_{without}=60). Median PFS with deletion 14 months, without 33 months. PFS Logrank p=8.59×10⁻⁴ (PFS N_{cell death deletion}=24, N_{without}=60).

FIG. 4. Schematic of the processes using homozygous deletiongs (HD) in cell death genes to identify the 97 gene signature.

FIG. 5. Survival curves for samples with altered expression of the 97 gene signature (lower curves, grey) against those without altered expression of the 97 gene signature (upper curves, black) in Myeloma IX. Top panel shows OS—Overall Survival (days), bottom panel shows PFS—Progression-Free survival (days). Median OS in altered expression cluster 33 months, others 48 months. OS Logrank p=6.67×10⁻³ (N_{cell death cluster}=85, Nₒₜₕₑᵣₛ=173). Median PFS in altered expression cluster 15 months, others 23 months. PFS Logrank p=0.0357 (N_{cell death cluster}=85, Nₒₜₕₑᵣₛ=173).

FIG. 6. Survival curves for samples with altered expression of the 97 gene cell death signature (lower curves, grey) against those without altered expression of the 97 gene signature (upper curves, black), in GSE9782. Top panel shows OS—Overall Survival (months), bottom panel shows PFS—progression-free survival (months). Median OS in altered expression cluster 8 months, others 21 months. OS Logrank p=2.66×10⁻⁹ (N_{cell death cluster}=18, Nₒₜₕₑᵣₛ=246). Median PFS in altered expression cluster 2 months, others 5 months. PFS Logrank p=0.00153 (N_{cell death cluster}=18, Nₒₜₕₑᵣₛ=246).

FIG. 7. Survival curves for samples with altered expression of the 97 gene cell death signature (lower curves, grey) against those without altered expression of the 97 gene signature (upper curves, black) in GSE2658. Panel shows OS—Overall Survival (months). Median OS in altered expression cluster 58 months, others >70 months. OS Logrank p=3.16×10⁻⁴ (N_{cell death cluster}=117, Nₒₜₕₑᵣₛ=442).

FIG. 8. Venn diagram showing the overlap of predictions between UAMS 70, IFM 15 and our 97 gene signature.

FIG. 9. Schematic of the process used to identify the six gene signature.

FIG. 10. Survival curves for samples positive for the six gene signature (lower curves, grey, “regressed”) against those negative for the six gene signature (upper curves, black, “not”) in Myeloma IX. OS—Overall Survival (days). Median OS with the signature 12 months, without 45 months. Logrank p=2.9×10⁻⁹ (N_{six gene sig}=17, Nₒₜₕₑᵣₛ=241).

FIG. 11. Survival curves for samples positive for the six gene signature (lower curves, grey, “regressed”) against those negative for the six gene signature (upper curves, black, “not”) in GSE9782 (top panel) and GSE2658 (bottom panel). OS—Overall Survival (months). Median OS in GSE9782 with signature 12 months, without 23 months. Logrank p=1.57×10⁻³ (N_{six gene sig}=108, Nₒₜₕₑᵣₛ=156). Median OS in GSE2658 with signature 46 months, without >70 months. Logrank p=2.94×10⁻⁸ (N_{six gene sig}=106, Nₒₜₕₑᵣₛ=453).

### DETAILED DESCRIPTION

### Multiple Myeloma

Multiple myeloma (also known as MM, myeloma, plasma cell myeloma, or as Kahler's disease) is a type of cancer of plasma cells which are immune system cells in bone marrow that produce antibodies. Myeloma is regarded as incurable, but remissions may be induced with steroids, chemotherapy, thalidomide and stem cell transplants. Myeloma is part of the broad group of diseases called hematological malignancies.

Treatment for multiple myeloma is focused on disease containment and suppression. If the disease is completely asymptomatic (i.e. there is a paraprotein and an abnormal bone marrow population but no end-organ damage), treatment may be deferred.

In addition to direct treatment of the plasma cell proliferation, bisphosphonates (e.g. pamidronate or zoledronic acid) are routinely administered to prevent fractures and erythropoietin to treat anaemia.

Initial treatment of multiple myeloma depends on the patient's age and comorbidities. In recent years, high-dose chemotherapy with hematopoietic stem-cell transplantation has become the preferred treatment for patients under the age of 65. Prior to stem-cell transplantation, these patients receive an initial course of induction chemotherapy. The most common induction regimens used today are thalidomide-dexamethasone, bortezomib based regimens, and lenalidomide-dexamethasone. Autologous stem cell transplantation, the transplantation of a patient's own stem cells after chemotherapy, is the most common type of stem cell transplantation for multiple myeloma. It is not curative, but does prolong overall survival. Allogeneic stem cell transplantation, the transplantation of a healthy person's stem cells into the affected patient, has the potential for a cure, but is only available to a small percentage of patients. Furthermore, there is a 5-10% treatment-associated mortality rate.

Patients over age 65 and patients with significant concurrent illness often cannot tolerate stem cell transplantation. For these patients, the standard of care has been chemotherapy with melphalan and prednisone. Recent studies among this population. suggest improved outcomes with new chemotherapy regimens. Treatment with bortezomib, melphalan and prednisone had an estimated overall survival of 83% at 30 months, lenalidomide plus low-dose dexamethasone an 82% survival at 2 years and melphalan, prednisone and lenalidomide had a 90% survival at 2 years. Head-to-head studies comparing these regimens have not been performed.

The present invention relates to methods for determining the prognosis for an individual having multiple myeloma. The results provided herein-demonstrate that altered expression of genes involved in cell death pathways is correlated with poor prognosis in multiple myeloma. In particular, the results provided herein demonstrate that altered expression patterns of genes belonging to a six gene signature of cell death genes are correlated with poor prognosis in multiple myeloma.

The determination of altered or dysregulated expression of cell death genes may involve determining the absolute or relative amount of mRNA for that gene in a sample (i.e. the amount of mRNA transcribed from that gene, which may be referred to as the amount of mRNA corresponding to that gene). Methods for doing this are well known to the skilled person. By way of example they include (i) using a labelled probe that is capable of hybridising to a cell death gene mRNA; (ii) using PCR involving one or more primers based on a cell death gene sequence, in particular using quantitative PCR methods; (iii) using commercially available or custom-built microarrays; (iv) Northern blotting; (v) serial analysis of gene expression (SAGE); and (vi) high throughput sequencing technologies.

Other methods of determining levels of gene expression are well known in the art and include methods of measuring amounts, concentrations or rates of synthesis of a protein encoded by the gene of interest. By way of example, such methods include (i) using a binding agent capable of specifically binding to a cell death protein, or a fragment thereof, in particular using an antibody capable of specifically binding the protein or fragment thereof. The antibody may be labelled to enable it to be detected or capable of detection following reaction with one or more further species, for example using a secondary antibody that is labelled or capable of producing a detectable result, e.g. in an ELISA type assay or Western blot; (ii) using immunohistochemical (IHC) analysis carried out on paraffin fixed samples or frozen tissue samples, this generally involves staining the samples to highlight the presence and location of protein.

Preferably, determination of altered expression of cell death genes is carried out by extracting RNA from a sample of myeloma cells, such as plasma cells from the bone marrow of an individual having multiple myeloma. The amount of RNA is then determined using PCR-based methods. Preferably quantitative PCR techniques using fluorigenic probes are used, as they are suitable for simultaneously analysing the expression of a plurality of different genes in a single sample. Such techniques are well known in the art, e.g. Taqman®-based techniques.

Preferably, the relative expression of each gene in a gene pair is determined, wherein a gene pair comprises a first gene selected from a high expressor cell death gene cluster, and a second gene selected from a low expressor cell death gene cluster (Table 1). Conveniently, RNA may be extracted from a sample of myeloma cells, which may be cells taken from a patient sample and then selected for CD 138+ cells, for example by immunomagnetic enrichment using commercially available magnetic beads linked to =anti-CD138 antibodies. The RNA is used to make cDNA by reverse transcription. An aliquot of this cDNA is then combined with Taqman® reagents—a set of primers and probes for each gene of the gene pair. The probes for each respective gene in the gene pair may be different colours, and thus amounts of cDNA for each gene can be measured independently in the same reaction. With such methods it is not necessary to use control genes, since each gene of the pair is in the same reaction. It is also not necessary to measure the absolute level of each of the genes, because the reactions are combined. This reduces complexity and the possibility of error.

Once the threshold value has been determined for the amplification reactions for both genes of the gene pair a cycle threshold value (Ct) for each gene can be determined. The Ct is the number of PCR cycles at which a significant exponential increase in fluorescence is detected, and which is directly correlated with the number of copies of DNA template present in the reaction. These Ct values can be used to determine the expression ratio of the genes.

Once the quantitative PCR reaction is optimized, examination of the amplification curve for each respective gene in the gene pair may be used to determine the results (i.e. the relative gene expression of each gene in a gene pair) without the step of calculating an expression ratio. For instance if a high expressor gene from the six gene signature (such as BUB1B) amplifies above a threshold value first (i.e. before its respective low expressor gene, in this case HDAC3) then there is more cDNA for the high expressor gene than for the low expressor gene in the sample i.e. a positive result, indicative of poor prognosis.

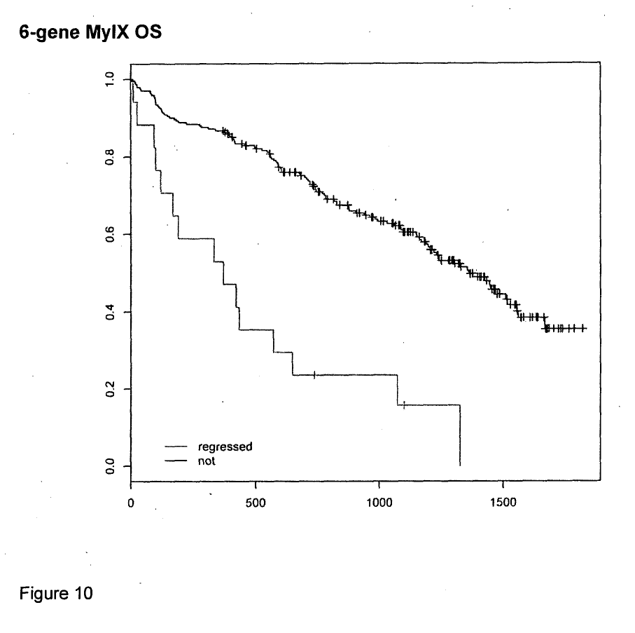

A prognosis obtained using the methods of the present invention may help to determine treatment of a myeloma patient. In particular, methods relating to the six gene signature identify high-risk patients, who have a poor prognosis regardless of therapies assessed (this includes different therapies in the validation data sets GSE9782 and GSE2658). In the Myeloma IX patient group the prognosis for patients positive for the six gene signature was much worse than those negative for the six gene signature (12 months median overall survival versus 45 months for those without) (FIG. 10).

Multiple myeloma patients positive for the six gene signature may respond better given a certain chemotherapy that those without. This can be determined by a randomized clinical trial. It is possible that for patients who are positive for the six gene signature, conventional chemotherapy or an intensive chemotherapy regime may not improve prognosis. In which case alternative treatments could be used such as auto-graft, where a patient's bone marrow blood stem cells are removed, and the patient is then given ablative chemotherapy, followed by return of their own stem cells. In addition, patients with a donor sibling or close match could be given an allograft bone marrow transplant. Patients who are positive for the six gene signature may have a poor prognosis regardless of therapies used, in which case determination of a negative result for the six gene signature may indicate that a patient's prognosis is more likely to improve in response to therapies such as conventional chemotherapy.

### Materials and Methods

### Patient Samples

Bone marrow aspirates were obtained from newly diagnosed patients with multiple myeloma, entered into the MRC myeloma IX study, after informed consent. The trial recruited 1970 patients and comprised two broad arms one for older and less fit patients and the other for younger fitter patients. All younger patients received autologous transplantation following induction with either CTD or CVAD. Older patients were treated with either CTD or MP. All patients were offered randomization to thalidomide maintenance or no maintenance. Patients in this analysis were representative of the trial in general and the trial patients behaved as would be expected.

Plasma cells were selected to a purity of >90% using CD138 microbeads and magnet-assisted cell sorting (Miltenyi Biotech, Bergisch Gladbach, Germany). Selected cells were split for analysis by FISH and for extraction of RNA and DNA. FISH was performed using standard approaches. RNA and DNA were extracted using commercially available kits (RNA/DNA mini kit or Allprep kit, Qiagen, Crawley, UK) according to manufacturers' instructions. Matched germline DNA from the 84 patients was also extracted from peripheral white blood cells, using the Flexigene kit (Qiagen).

### Genome Mapping and Expression Analysis

DNA and RNA were prepared for hybridization to the GeneChip Mapping 500K Array set and the U133 Plus 2.0 expression GeneChip, respectively (Affymetrix, Santa Clara, Calif.) according to manufacturers instructions, and have previously been described.^{3,5,10} Analysis of mapping array data were performed as previously described using GCOS, GTYPE, dChip and CNAG. The expression levels were generated using dChip (April 2007), using the default perfect match/mismatch calculations and median normalization.

### Copy Number Analysis

SNP genotypes were obtained using Affymetrix GCOS software (version 1.4) to obtain raw feature intensity which was then processed using Affymetrix GTYPE software (version 4.0) to derive SNP genotypes. Multiple samples were analyzed together using output from GCOS and GTYPE using dChip.¹¹ The matched control samples were assigned a copy number of two and used as a reference set to calculate copy number in tumor samples. Median smoothing with a window size of 11 was used to infer copy number along each chromosome. All results were verified using outputs from CNAG.¹² To increase the accuracy of detection of acquired HDels in the tumor we also performed 500K mapping arrays on paired peripheral blood DNA from the same patients. This approach minimizes copy-number variation between samples, and reduces false positive deletions which may be present in the germ-line DNA that are not acquired in the tumor. The paired tumor-control copy number data were inferred using dChip (April 2007).¹¹ The data were generated using the default median smoothing method with an 11 SNP window.

### Homozygous Deletion Analysis

A dChip inferred copy number threshold value of 0.93 was used to identify Homozygous deletions (HDels). This number was derived by looking at the distribution of inferred copy number values for a range of SNPs across chromosome 13, in 19 cases where FISH indicated monosomy of chromosome 13. This distribution was used to identify the lower boundary of HDels (copy number 1). Anything falling below the lower boundary, by definition, have less than 1 copy and so be a HDel. The lower boundary was defined as the bottom 1% of the distribution, which was calculated as 0.93, therefore, anything below 0.93 should be an HDel. The dChip method used is highly sensitive but tends to over-predict copy number changes.¹³ In order to compensate, in part, for this sensitivity we defined a deletion as having to be at least two SNPs below the threshold level. In order to maintain some sensitivity we added a window of 3 times the median distance between SNPs in the genome (5.8 kb on the 500K array). This 17.4 kb window allows intervening SNPs to go above the threshold as long as they are surrounded by SNPs that are deleted (<0.93). Comparisons between data sets that both allowed and rejected regions of deletion that had intervening SNPs showed that having the window increases sensitivity to regions that are known to have HDels in myeloma. HDels were further filtered by integration with expression array data to identify HDels which affect expression of genes,

### Survival Analysis

Kaplan-Meier survival curves calculated using Bioconductor and the survival package.¹⁴ The difference between curves was tested using the Logrank test again using R. A threshold of significance was taken as p<0.05. The relationship between common prognostic factors such as B2M, age, t(4;14) and del(13) and survival has been analysed using univariate Cox regression. In addition, the independence of these factors and the HR signature was compared using multivariate Cox regression and a two-stage filtering process.

### FISH

FISH was performed using standard approaches aimed at identifying translocation partners (t(4;14), n=7; t(6:14), n=2; t(8;14), n=1; t(11;14), n=11; t(14;16), n=4; t(14;20), n=2; and 7 cases with split 14 but unidentified partners) and hyperdiploidy (n=42, of which 9 also have a split 14) by examining chromosomes 3, 4, 5, 7, 9, 11, 13, 14, 15 and 17 using previously described probes². Hyperdiploidy was defined primarily on the results of chromosomes 5, 9 and 15 but modified by the results from other probes used.^{19,20} An additional 6 cases had no abnormalities using these probes and 11 cases do not have FISH data.

### Annotating and Filtering Homozygous Deletions

All of the annotation of SNP locations and identification of genes were performed using Ensembl 48 mappings, which is based on the genome assembly NCBI36 and dbSNP127. A gene was assigned to a SNP if the location of, the SNP was anywhere in the gene region, including all intronic, exonic and UTR regions. Every probeset for a gene was used for filtering purposes. To pass the filtering step the probeset had to pass three criteria. Initially, genes must be expressed in more then 1% of myeloma cases. An expression threshold was defined as having a signal intensity of at least 250 in our dataset. This threshold was derived by looking at a gene which is not expressed in a subset of myeloma cases, MMSET. In cases with a t(4;14) translocation it is highly expressed and in cases without the translocation it is not expressed. Looking at a graph of expression and corresponding FISH data, the cases without expression are easily identifiable and the threshold was taken as the maximum of non-expression for this gene. Again several thresholds were tried but this proved to be the most robust (data not shown). The second criterion was that HDel should ablate expression of the gene if it is having a significant effect which the expression arrays can pick up. A threshold of 150 was set in order to be conservative about our estimate of HDel (again several thresholds were tried and this was the most robust). Expression in the deleted cases should be lower than this threshold, and cases were rejected if expression exceeded this value. Lastly, to ensure that these HDels are relevant to the pathogenesis of myeloma, and must be present with loss of expression in at least 2 samples. This was carried out in all probesets for a gene, since it is difficult to automatically decide which probeset is the best for that gene. In order to maintain sensitivity when filtering, probesets that passed the criteria were chosen over those that failed and the probeset that produced the highest proportion of passes for any HDel was picked as the representative probeset.

Confirmation of DNA Copy Number by qPCR

The copy number of homozygously deleted regions was validated on myeloma sample DNA by qPCR. qPCR was performed and normalized to the PRKCQ locus as previously described,²¹ using a 7500 Fast Real-Time PCR System (Applied Biosystems) with Power SYBR Green PCR master mix (Applied Biosystems). Two peripheral blood DNA samples were used as calibrators. 3 regions were picked to confirm HDels across the dataset which encompassed a broad spectrum of copy numbers from 0.31 to 0.9. Additional samples which were not detected as having HDels were also included in the assay. These regions included the deletion at CDKN2C (For 5′-TTGTTCAGTTTCACTGAATATAAAGAAGTG-3′, Rev 5′-GCCAGCCTATTTTCATTTAAGGAA-3′) and BIRC3 (For 5′-CAGTCCCCCAAAGCCTTACC-3′, Rev 5′-TCAGATGTGACTTTGGGACTTTTGATT-3′), located near SNPs rs6588394 and rs11225203, respectively.

### GO Classification

There were few genes deleted in more than 5% of myeloma cases and none in more than 16% of cases meaning that determination of the effects of HDel on PFS and OS lacked power. In order to assess the impact of HDels acting at different points within a pathway on OS and PFS, genes were grouped into pathways using a knowledge-driven approach based on automatic annotation of the genes with HDels. The annotation set with the most complete and informative coverage of our data was Gene Ontology (GO) terms (we also looked at Biocarta and KEGG pathways).^{22,23} We used a GO tree level of 5 because this provides more detailed and specific information about the function of the genes while still providing good annotation coverage. We applied the annotations using DAVID 2008 because it assigned more annotations to our genes than Ensembl 48 GO annotations.²⁴ When selecting from the list we did not use the measures of significance of over representation from DAVID as criteria only the GO term annotation. We then looked for significant differences in overall survival between those that had deletions in genes annotated in a particular GO term and those without these HDels. We only examined GO terms that were informative and biologically relevant.

### Results

### Homozygous Deletions in Presenting Patient Samples

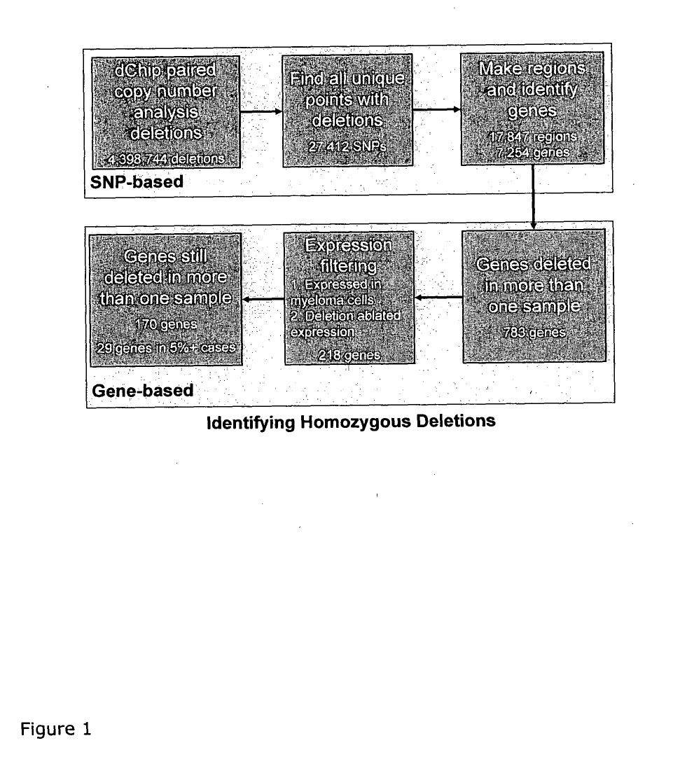

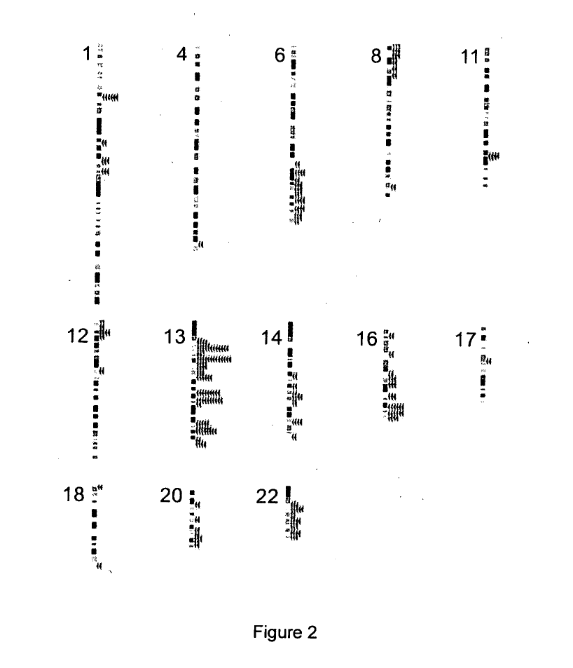

Mapping data from 84 newly diagnosed myeloma patients (Table 2) were analyzed for the presence of homozygous deletions. We identified 27,412 SNPs with deletion in any sample using a cut-off of 0.93, which equates to 4,398,744 homozygous deletion points across the 84 samples, or 5.4% of the genome. After removal of deletions constituting a single SNP, these deletions result in 18,733 genomic regions, or 7,254 genes. After filtering to remove genes present in only 1 sample, 783 genes contain a homozygous deletion. Using the criteria outlined in the methods described below to link homozygous deletion to the expression of genes contained within the region, 218 unique genes were identified in which the deletion affects expression of the gene. Of these genes, 170 were present in more than one sample, and 29 genes were present in more than 5% of samples (FIG. 1). When these 29 genes are mapped to their genomic locations (FIG. 2) they are found predominantly in the regions previously identified as frequently being involved in hemizygous deletions (1p, 6q, 11q, 12p, 13q, 14q, 16q and 22q). Of the 29 homozygously deleted genes, 14 were located on chromosome 13. A subset of HDels was confirmed by qPCR.

Survival analyses were carried out an all of the genes with deletions in ≧5% of the cases. Of these genes, deletion of 1p32 at CDKN2C/FAF1 has a significant effect on overall survival (OS) (p=0.029), median OS 20 months with and 46 months without. There were five genes deleted in at least 10% of cases DCLK1, ATP8A2, KLF12, PCDH9 and FGF14. Of these only ATP8A2 had a significantly worse OS (p=0.018), median OS of 14 months with and 46 months without.

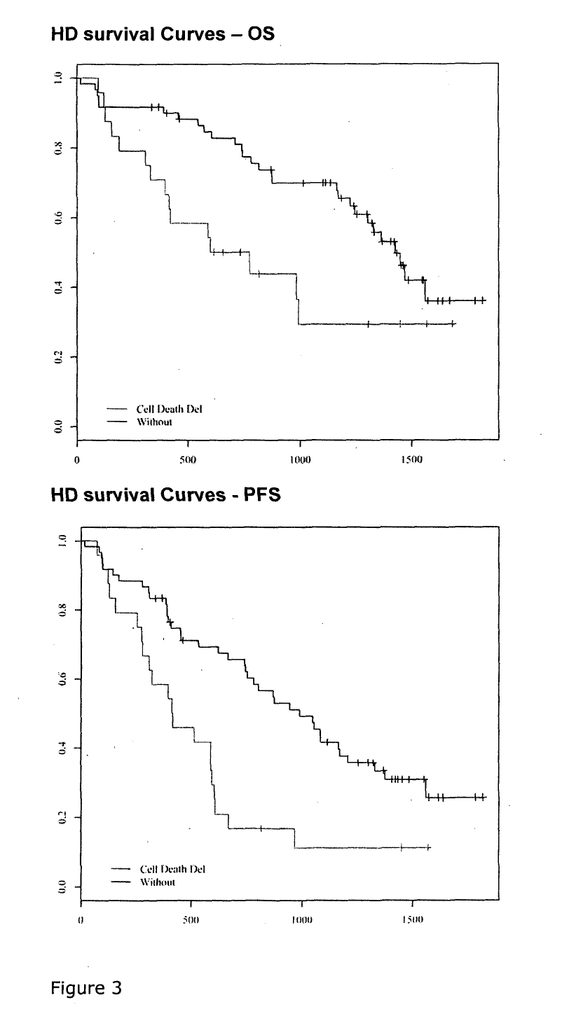

Gene Ontology (GO) annotations were used to interpret the identified genes and define pathway-specific abnormalities present within the filtered 170 gene list. There were 43 terms at GO level 5 assigned to the list and after consideration of the annotations we tested the prognostic significance of the following GO terms: regulation of progression through cell cycle probeset (GO:0000074), negative regulation of progression through cell cycle (GO:0045786), protein transport (GO:0015031), intracellular transport (GO:0046907) and cell death (GO:0008219). In this analysis samples with a deletion of any gene annotated as cell death (n=25 26/68%) had a significantly worse OS (p=2.36×10⁻³) and PFS (p=9.83×10⁻³) median OS with 26 months and 47 months without (FIG. 3). These cell death genes were FAF1, CDKN2C, CTSB, TNFRSF10B, TNFRSF10D, BIRC3, ESR1, PLAGL1, SGK, EMP1, FGF14, FOXO1, TFDP1, and KRT18. There was no over-representation of t(4;14), t(14;16), t(14;20), high P2M, del(16q) or del(17p) within the cell death deleted cases compared to all of the samples. There was, however, an over-representation of del of 13 (p=0.037) but these cases do not have a significantly worse OS than those without, therefore, it is not the effect of dell3 that we are seeing. Examination of a proliferation index (PI) within the cell death deleted cases shows that there was an over-representation of high PI samples (p=0.019). However, removal of cases with a high PI reveals that the residual cases still have a significantly worse overall survival (p=0.029) indicating that the cell death pathway is an independent prognostic factor.

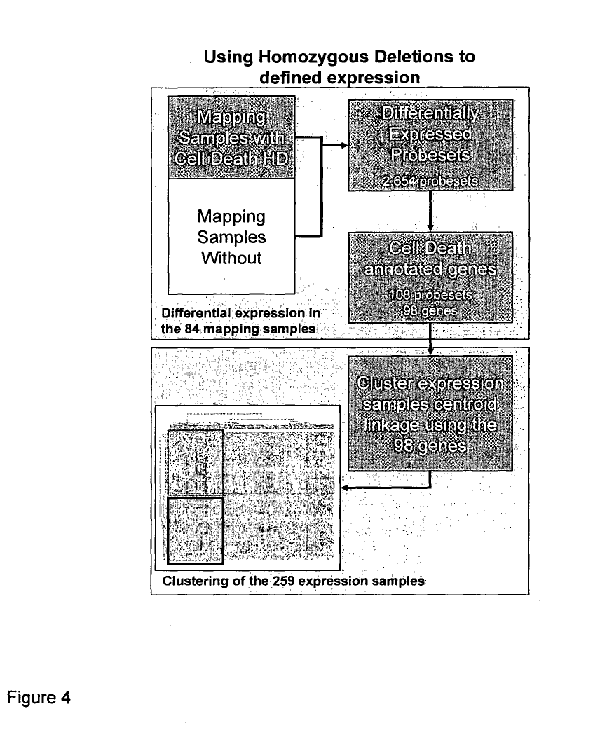

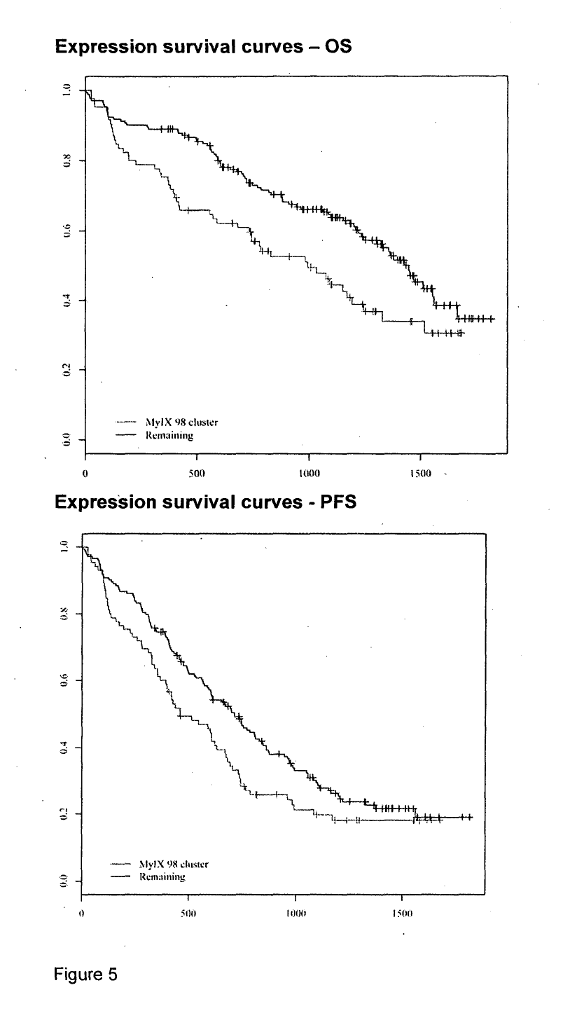

We wished to translate this prognostic finding based on changes at the DNA level into a readily utilisable signature based on related gene expression. Supervised hierarchical clustering was used to identify genes that are differentially expressed (p<0.05) between samples with and without homozygous deletion of a cell death gene. This analysis generated a list of 2,654 genes, of which 97 were annotated in the Cell Death GO term (FIG. 4, Table 1). Unsupervised hierarchical clustering (centroid-based) was performed on 259 samples using the 97 gene list and revealed 2 clusters with unique expression signatures. The Bioconductor package pamr was used to confirm the signature and determine if the gene list was sufficient to classify the samples. The samples with altered expression in the cell death network had a significantly worse OS, median 33 months with deletion and 48 without deletion, and worse PFS (FIG. 5). The samples in this cell death expression class were checked for an over-representation of other known factors that would affect survival and they were representative of our set as a whole.

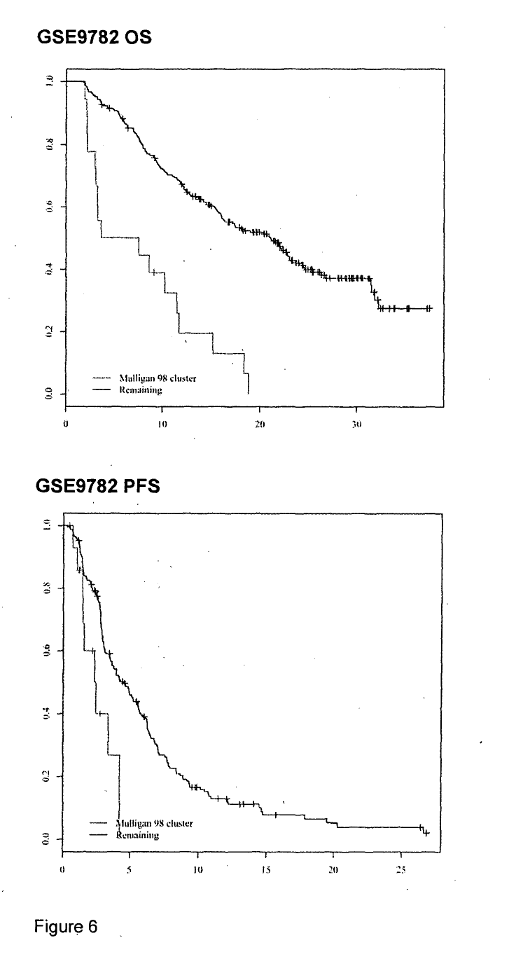

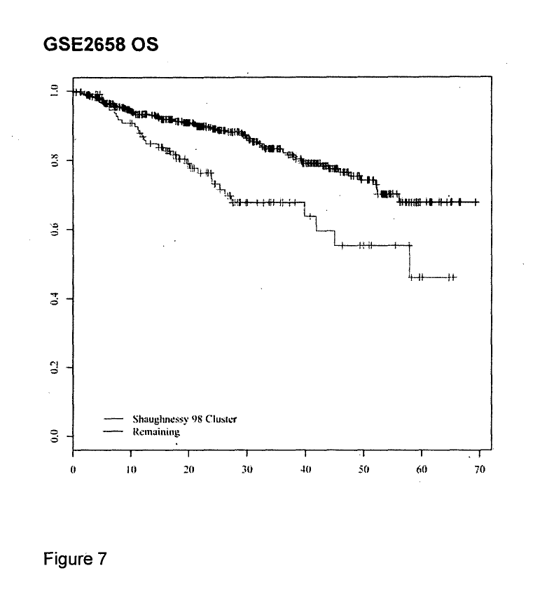

In order to validate this signature we repeated the analysis in two additional datasets (GSE2658 and GSE9782) and the same cluster was identified in each of these datasets. In both data sets the cell death dysregulated cases had a significantly worse overall survival GSE2658 (FIGS. 6 and 7). The GSE2658 data set comprised patients derived from patients in the total therapy programmes TT2 and TT3 the first using thalidomide and the second thalidomide plus bortezomib. The GSE9782 data set comprised relapsed patients treated with bortezomib from the CREST, SUMMIT and APEX studies including the 040 companion study.

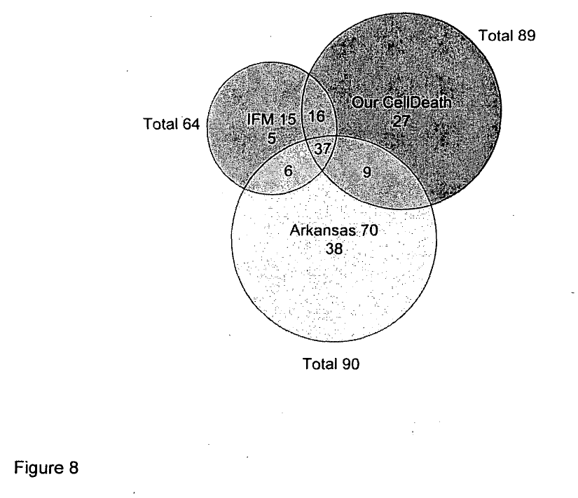

We applied two other published prognostic signatures (UAMS 70 gene and IFM 15 genes) on our data set (FIG. 8). The genes in each of the signatures are different with no overlap apart from BIRC5. There was a core group of cases identified by each of the signatures, however, our signature identifies a distinct set of poor prognosis cases, and is more sensitive than the IFM 15 signature and equally sensitive as the UAMS 70 gene signature. Multivariate Cox regression confirms that our signature is a significant independent variable in determining overall survival when compared with the predictions of the IFM 15 and the UAMS 70 signatures.

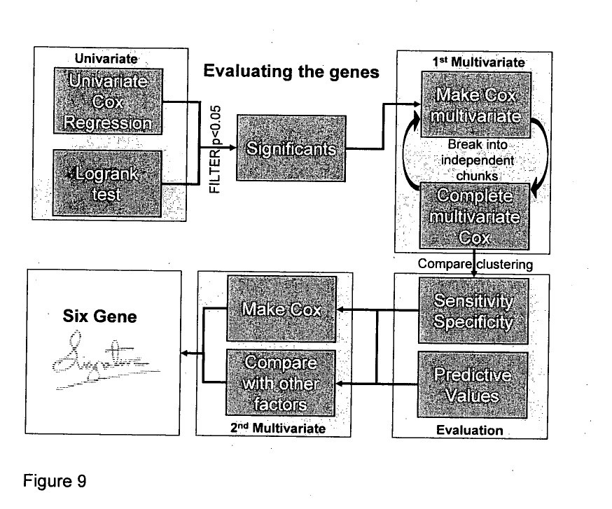

As shown schematically in FIG. 9, the six gene signature was then developed from the 97 gene signature. Hierarchical clustering showed that there were two groups of expression in the 97 gene signature, the relatively high expressors and the relatively low expressors. For every pair of high versus low expressors the ratio of expression was calculated for each of the 259 samples. Then for each gene pair the samples were split into groups of high ratio (>=1) and low ratio (<1). Then survival tests were performed using these two groups. A univariate analysis was performed using both logrank tests and Cox regression (a ratio is used to divide the samples into two groups for simplicity, as the logrank test requires groups; the Cox test is a more continuous ratio versus survival test). A threshold of p<0.05 was taken to filter the results. Pairs that were significant in the univariate analysis were grouped together into independent groups and used in a multivariate analysis to determine the independent predictors of survival. A process of trimming and repeating was performed until a smaller, converging set of groups remained. (For those pairs with duplicate probesets the probeset with the biggest effect was selected, using the univariate Cox r² values.) The best three pairs of genes were chosen as prognostic markers and used to classify our data and both of the validation data sets. Cases were assigned as high risk classification if any one of the three pairs has high ratio (FIG. 9). (The probesets used were also assessed on sensitivity, specificity, positive predictive value and negative predictive value.) The predictions made by using these genes was also analysed in a multivariate Cox regression to compare with other known prognostic factors, including B2M, age, t(4;14) status, del(13) status (Table 3).

### Discussion

Homozygous deletions have been shown to affect genes important in tumour progression and clinical outcome.¹⁵⁻¹⁸ In this work on myeloma the present inventors show that the frequency of HDels is variable, with 56 out of 84 cases having at least 1 HDel, (maximum 64, median 5.5 HDel per case). These HDels tend to occur in the genomic regions where hemizygous deletions occur: 1p, 6q, 8p, 13q, 16q, 20p and 22 but in addition, frequent recurrent HDels are also seen at 11q, 12p and 14q results that are consistent with and extend further other results in the literature.^{18,15,40}. Of the genes that were deleted in at least 10% of cases only ATP8A2 had a significantly worse OS (p=0.018) median 14 months with and 46 months without.

HDels relevant to myeloma pathogenesis were identified by screening for HDels which occurred twice or more, in genes expressed in plasma cells with decreased expression in the presence of a HDel. This approach identified 170 genes and the inventors identified a significant enrichment of genes within the GO defined cell death pathways. The term cell death includes cytolysis and programmed cell death, programmed cell death and is an umbrella term that includes both apoptotic and non-apoptotic programmed cell death. In this analysis the genes significantly enriched within this term included genes important in cell cycle regulation (CDKN2C, EMP1, PLAGL1), apoptosis (CTSB, BIRC2, BIRC3, TNFRSF10B, TNFRSF10D, FAF1, FGF14, SGK), and regulation of transcription (ESR1, FOXO1, TFDP1). While there were 15 genes in the cell death signature there were only 11 distinct genetic regions, this difference being due to juxtaposed pairs of genes being deleted in the same cases: CDKN2C and FAF1 on 1p, SGK and ESR1 on 6q, TNFRSF10B and TNFRSF10D on 8p, and BIRC2 and BIRC3 on 11q. Deletion within any one of the genes within the GO defined cell death pathway identified 25% of all cases of myeloma and was linked with impaired OS and PFS that was not due to a co-segregating factor.

The cases with a dysregulated cell death network at a DNA level were used as a test set to identify associated expression changes within genes included within the same GO terms. This identified a signature consisting of 97 genes associated with poor outcome (Table 1). The content of the list has an overrepresentation of genes within the intrinsic and extrinsic apoptotic pathway as well as within the TRAIL/TNF/NFkB signalling pathway and PI3k pathway. This 97 gene signature identifies cases with a poor prognosis (OS PFS medians) suitable for alternate treatment strategies. The proportion of cases identified at 25% is a fraction that would be suitable for alternate strategies. We tested the signature for its validity at presentation, relapse and by treatment used including thalidomide or bortezomib. It was an independent prognostic factor in each of these settings. The signature is not predictive of response to a particular therapy. We compared it use to two other published signatures and found it was independent of the DAMS signature.

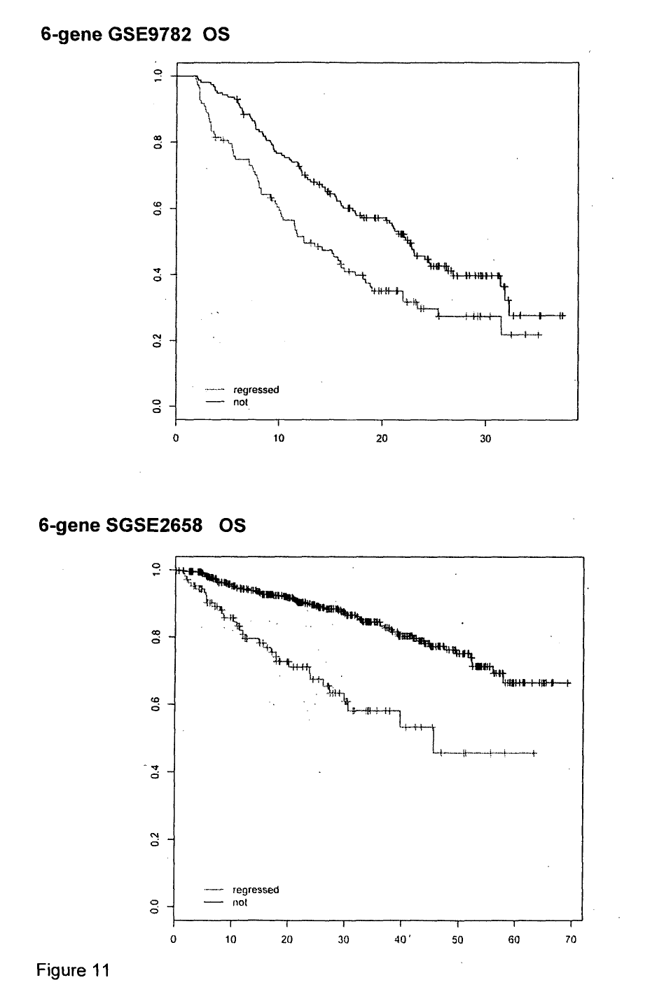

The use of global gene expression brings challenges within the clinical environment and consequently we developed a more limited signature which could give similar information. This list utilised information from 3 pairs of genes suitable for RQPCR analysis (real time quantitiative PCR analysis). This approach based on BUB1B/HDAC3, CDC2/FIS1 and RAD21/ITM2B has a sensitivity and specificity for identification of cases identified by the 97 gene signature. Cases identified by this signature have median OS of 12 months, compared to 45 months for cases without this signature (FIG. 10). The prognostic value of the six gene signature was confirmed in the independent data sets GSE9782 and GSE2658 (FIG. 11). Important prognostic indicators for myeloma include the ISS, 17p- and a number of poor prognosis Ig translocation but all suffer from poor sensitivity and specificity for identifying poor risk cases (Table 4). In this work we have developed a limited signature which has high sensitivity and specificity for the identification of poor risk cases at presentation and relapse which are suitable for alternate treatment approaches.

The documents disclosed herein are all expressly incorporated by reference in their entirety.

TABLE 1

| | | | | | | Ex- |
|---|---|---|---|---|---|---|
| | Entrez | | Chromo- | | | pressor |
| Gene | gene id | Description | some | Band | Probeset | Class |
| TNFRSF14 | 8764 | Tumor necrosis factor receptor superfamily member 14 precursor (Herpesvirus | 1 | p36.32 | 209354_at | low |
| | | entry mediator A) (Tumor necrosis factor receptor-like 2) (TR2). | | | | |
| | | [Source: Uniprot/SWISSPROT; Acc: Q92956] | | | | |
| UBE4B | 10277 | Ubiquitin conjugation factor E4 B (Ubiquitin fusion degradation protein 2) | 1 | p36.22 | 202316_x_at | high |
| | | (Homozygously deleted in neuroblastoma 1). | | | | |
| | | [Source: Uniprot/SWISSPROT; Acc: O95155] | | | | |
| HDAC1 | 3065 | Histone deacetylase 1 (HD1). [Source: Uniprot/SWISSPROT; Acc: Q13547] | 1 | p35.1 | 200644_at | high |
| MARCKSL1 | 65108 | MARCKS-related protein (MARCKS-like protein 1) (Macrophage | 1 | p35.1 | 200644_at | high |
| | | myristoylated alanine-rich C kinase substrate) (Mac-MARCKS) | | | | |
| | | (MacMARCKS). [Source: Uniprot/SWISSPROT; Acc: P49006] | | | | |
| RRAGC | 64121 | Ras-related GTP-binding protein C (Rag C) (GTPase-interacting protein 2) | 1 | p34.3 | 218088_s_at | low |
| | | (TIB929). [Source: Uniprot/SWISSPROT; Acc: Q9HB90] | | | | |
| UTP11L | 51118 | Probable U3 small nucleolar RNA-associated protein 11 (U3 snoRNA- | 1 | p34.3 | 218235_s_at | high |
| | | associated protein 11) (UTP11-like protein). [Source: | | | | |
| | | Uniprot/SWISSPROT; Acc: Q9Y3A2] | | | | |
| FAF1 | 11124 | FAS-associated factor 1 (Protein FAF1) (hFAF1). | 1 | p33 | 218080_x_at, | low |
| | | [Source: Uniprot/SWISSPROT; Acc: Q9UNN5] | | | 224217_s_at | |
| DEDD | 9191 | Death effector domain-containing protein (Death effector domain- containing | 1 | q23.3 | 202891_at | high |
| | | testicular molecule) (DEDPro1) (FLDED-1). | | | | |
| | | [Source: Uniprot/SWISSPROT; Acc: O75618] | | | | |
| NIT1 | 4817 | Nitrilase homolog 1 (EC 3.5.—.—). [Source: Uniprot/SWISSPROT; Acc: | 1 | q23.3 | 202891_at | high |
| | | Q86X76] | | | | |
| SLAMF7 | 57823 | SLAM family member 7 precursor (CD2-like receptor activating cytotoxic | 1 | q23.3 | 219159_s_at | low |
| | | cells) (CRACC) (Protein 19A) (CD2 subset 1) (Novel Ly9) (Membrane protein | | | | |
| | | FOAP-12) (CD319 antigen). [Source: Uniprot/SWISSPROT; Acc: Q9NQ25] | | | | |
| EIF2AK2 | 5610 | Interferon-induced, double-stranded RNA-activated protein kinase (EC | 2 | p22.2 | 228495_at | high |
| | | 2.7.11.1) (Interferon-inducible RNA-dependent protein kinase) (Eukaryotic | | | | |
| | | translation initiation factor 2-alpha kinase 2) (eIF-2A protein kinase | | | | |
| | | 2) (Protein kinase RNA-activated) (PKR) ([Source: Uniprot/SWISSPROT; | | | | |
| | | Acc: P19525] | | | | |
| FBXO11 | 80204 | F-box only protein 11 (Vitiligo-associated protein VIT-1). | 2 | p16.3 | 202911_at | high |
| | | [Source: Uniprot/SWISSPROT; Acc: Q86XK2] | | | | |
| MSH6 | 2956 | DNA mismatch repair protein MSH6 (MutS-alpha 160 kDa subunit) (G/T | 2 | p16.3 | 202911_at | high |
| | | mismatch-binding protein) (GTBP) (GTMBP) (p160). | | | | |
| | | [Source: Uniprot/SWISSPROT; Acc: P52701] | | | | |
| RTN4 | 57142 | Reticulon-4 (Neurite outgrowth inhibitor) (Nogo protein) (Foocen) | 2 | p16.1 | 214629_x_at | low |
| | | (Neuroendocrine-specific protein) (NSP) (Neuroendocrine-specific protein | | | | |
| | | C homolog) (RTN-x) (Reticulon-5). [Source: Uniprot/SWISSPROT; | | | | |
| | | Acc: Q9NQC3] | | | | |
| TIA1 | 7072 | Nucleolysin TIA-1 isoform p40 (RNA-binding protein TIA-1) (p40-TIA-1) | 2 | p13.3 | 201448 at, | high, |
| | | [Contains: Nucleolysin TIA-1 isoform p15 (p15-TIA-1)]. | | | 201450_s_at | high |
| | | [Source: Uniprot/SWISSPROT; Acc: P31483] | | | | |
| STAMBP | 10617 | STAM-binding protein (EC 3.1.2.15) (Associated molecule with the SH3 | 2 | p13.1 | 202811_at | high |
| | | domain of STAM). [Source: Uniprot/SWISSPROT; Acc: O95630] | | | | |
| ALS2CR12 | 130540 | Amyotrophic lateral sclerosis 2 chromosomal region candidate gene 12 | 2 | q33.1 | 213373_s_at | low |
| | | protein. [Source: Uniprot/SWISSPROT; Acc: Q96Q35] | | | | |
| CASP8 | 841 | Caspase-8 precursor (EC 3.4.22.61) (CASP-8) (ICE-like apoptotic protease 5) | 2 | q33.1 | 213373_s_at | low |
| | | (MORT1-associated CED-3 homolog) (MACH) (FADD-homologous | | | | |
| | | ICE/CED-3-like protease) (FADD-like ICE) (FLICE) (Apoptotic cysteine | | | | |
| | | protease) (Apoptotic protease Mch-5) (CAP4) [Contains [Source: | | | | |
| | | Uniprot/SWISSPROT; Acc: Q14790] | | | | |
| HSPE1 | 3336 | 10 kDa heat shock protein, mitochondrial (Hsp10) (10 kDa chaperonin) | 2 | q33.1 | 205133_s_at | high |
| | | (CPN10) (Early-pregnancy factor) (EPF). [Source: | | | | |
| | | Uniprot/SWISSPROT; Acc: P61604] | | | | |
| FASTKD2 | 22868 | FAST kinase domain-containing protein 2. | 2 | q33.3 | 216996_s_at | high |
| | | [Source: Uniprot/SWISSPROT; Acc: Q9NYY8] | | | | |
| NDUFS1 | 4719 | NADH-ubiquinone oxidoreductase 75 kDa subunit, mitochondrial precursor | 2 | q33.3 | 203039_s_at | high |
| | | (EC 1.6.5.3) (EC 1.6.99.3) (Complex I-75kD) (CI-75kD). | | | | |
| | | [Source: Uniprot/SWISSPROT; Acc: P28331] | | | | |
| BARD1 | 580 | BRCA1-associated RING domain protein 1 (BARD-1). | 2 | q35 | 205345_at | high |
| | | [Source: Uniprot/SWISSPROT; Acc: Q99728] | | | | |
| IL8RB | 3579 | High affinity interleukin-8 receptor B (IL-8R B) (CXCR-2) (GRO/MGSA | 2 | q35 | 207008_at | low |
| | | receptor) (IL-8 receptor type 2) (CD182 antigen) (CDw128b). | | | | |
| | | [Source: Uniprot/SWISSPROT; Acc: P25025] | | | | |
| CUL3 | 8452 | Cullin-3 (CUL-3). [Source: Uniprot/SWISSPROT; Acc: Q13618] | 2 | q36.2 | 201370_s_at | high |
| AXUD1 | 64651 | Axin-1 up-regulated gene 1 protein (TGF-beta-induced apoptosis protein 3) | 3 | p22.2 | 225557_at | low |
| | | (TAIP-3) (Protein URAX1). [Source: Uniprot/SWISSPROT; Acc: Q96S65] | | | | |
| GPX1 | 2876 | Glutathione peroxidase 1 (EC 1.11.1.9) (GSHPx-1) (GPx-1) (Cellular | 3 | p21.31 | 200736_s_at | low |
| | | glutathione peroxidase). [Source: Uniprot/SWISSPROT; Acc: P07203] | | | | |
| RHOA | 387 | Transforming protein RhoA precursor (H12). | 3 | p21.31 | 200736_s_at | low |
| | | [Source: Uniprot/SWISSPROT; Acc: P61586] | | | | |
| RYBP | 23429 | RING1 and YY1-binding protein (Death effector domain-associated factor) | 3 | p13 | 201844_s_at, | low |
| | | (DED-associated factor) (YY1 and E4TF1-associated factor 1) (Apoptin- | | | 201846_s_at | |
| | | associating protein 1) (APAP-1). [Source: Uniprot/SWISSPROT; | | | | |
| | | Acc: Q8N488] | | | | |
| RNF7 | 9616 | RING-box protein 2 (Rbx2) (RING finger protein 7) (Regulator of cullins 2) | 3 | q23 | 224395 s at, | low |
| | | (CKII beta-binding protein 1) (CKBBP1) (Sensitive to apoptosis gene | | | 224439_x_at | |
| | | protein). [Source: Uniprot/SWISSPROT; Acc: Q9UBF6] | | | | |
| PDCD10 | 11235 | Programmed cell death protein 10 (TF-1 cell apoptosis-related protein 15) | 3 | q26.1 | 210907_s_at | high |
| | | (Cerebral cavernous malformations 3 protein). [Source: | | | | |
| | | Uniprot/SWISSPROT; Acc: Q9BUL8] | | | | |
| BCL6 | 604 | B-cell lymphoma 6 protein (BCL-6) (Zinc finger protein 51) (LAZ-3 protein) | 3 | q27.3 | 203140_at | low |
| | | (BCL-5) (Zinc finger and BTB domain-containing protein 27). | | | | |
| | | [Source: Uniprot/SWISSPROT; Acc: P41182] | | | | |
| ANXA5 | 308 | Annexin A5 (Annexin-5) (Annexin V) (Lipocortin V) (Endonexin II) | 4 | q27 | 200782_at | high |
| | | (Calphobindin I) (CBP-I) (Placental anticoagulant protein I) (PAP-I) | | | | |
| | | (PP4) (Thromboplastin inhibitor) (Vascular anticoagulant-alpha) | | | | |
| | | (VAC- alpha) (Anchorin CII). [Source: Uniprot/SWISSPROT; Acc: P08758] | | | | |
| FASTKD3 | 79072 | FAST kinase domain-containing protein 3. | 5 | p15.31 | 219200_at | high |
| | | [Source: Uniprot/SWISSPROT; Acc: Q14CZ7] | | | | |
| PRKAA1 | 5562 | 5′-AMP-activated protein kinase catalytic subunit alpha-1 (EC 2.7.11.1) | 5 | p13.1 | 209799_at | low |
| | | (AMPK alpha-1 chain). [Source: Uniprot/SWISSPROT; Acc: Q13131] | | | | |
| XRCC4 | 7518 | DNA-repair protein XRCC4 (X-ray repair cross-complementing protein 4). | 5 | q14.2 | 205071_x_at, | low |
| | | [Source: Uniprot/SWISSPROT; Acc: Q13426] | | | 210813_s_at | |
| RASA1 | 5921 | Ras GTPase-activating protein 1 (GTPase-activating protein) (GAP) (Ras p21 | 5 | q14.3 | 202677_at, | high, |
| | | protein activator) (p120GAP) (RasGAP). | | | 210621_s_at | high |
| | | [Source: Uniprot/SWISSPROT; Acc: P20936] | | | | |
| HDAC3 | 8841 | Histone deacetylase 3 (HD3) (RPD3-2) (SMAP45). | 5 | q31.3 | 216326_s_at | low |
| | | [Source: Uniprot/SWISSPROT; Acc: O15379] | | | | |
| CYFIP2 | 26999 | Cytoplasmic FMR1-interacting protein 2 (p53-inducible protein 121). | 5 | q33.3 | 220999_s_at | high |
| | | [Source: Uniprot/SWISSPROT; Acc: Q96F07] | | | | |
| RNF130 | 55819 | Goliath homolog precursor (RING finger protein 130). | 5 | q35.3 | 217865_at | low |
| | | [Source: Uniprot/SWISSPROT; Acc: Q86XS8] | | | | |
| TNFRSF21 | 27242 | Tumor necrosis factor receptor superfamily member 21 precursor (TNFR- | 6 | p12.3 | 218856_at | high |
| | | related death receptor 6) (Death receptor 6). [Source: | | | | |
| | | Uniprot/SWISSPROT; Acc: O75509] | | | | |
| FOXO3 | 2309 | Forkhead box protein O3A (Forkhead in rhabdomyosarcoma-like 1) (AF6q21 | 6 | q21 | 204131_s_at | low |
| | | protein). [Source: Uniprot/SWISSPROT; Acc: O43524] | | | | |
| TPD52L1 | 7164 | Tumor protein D53 (HD53) (Tumor protein D52-like 1). | 6 | q22.31 | 203786_s_at | high |
| | | [Source: Uniprot/SWISSPROT; Acc: Q16890] | | | | |
| CYCS | 54205 | Cytochrome c. [Source: Uniprot/SWISSPROT; Acc: P99999] | 7 | p15.3 | 208905_at | high |
| FIS1 | 51024 | Mitochondrial fission 1 protein (Fis1 homolog) (hFis1) (Tetratricopeptide | 7 | q22.1 | 218034_at | low |
| | | repeat protein 11) (TPR repeat protein 11). [Source: Uniprot/SWISSPROT; | | | | |
| | | Acc: Q9Y3D6] | | | | |
| ZC3HC1 | 51530 | Nuclear-interacting partner of ALK (Nuclear-interacting partner of anaplastic | 7 | q32.2 | 223163_s_at | high |
| | | lymphoma kinase) (hNIPA) (Zinc finger C3HC-type protein 1). | | | | |
| | | [Source: Uniprot/SWISSPROT; Acc: Q86WB0] | | | | |
| CASP2 | 835 | Caspase-2 precursor (EC 3.4.22.55) (CASP-2) (ICH-1 protease) (NEDD2 | 7 | q34 | 226032_at | high |
| | | protein) (Neural precursor cell expressed developmentally down- regulated | | | | |
| | | protein 2) (NEDD-2) [Contains: Caspase-2 subunit p18; Caspase-2 | | | | |
| | | subunit p13; Caspase-2 subunit p12]. [Source: Uniprot/SWISSPROT; | | | | |
| | | Acc: P42575] | | | | |
| CUL1 | 8454 | Cullin-1 (CUL-1). [Source: Uniprot/SWISSPROT; Acc: Q13616] | 7 | q36.1 | 207614_s_at | high |
| DNAJB6 | 10049 | DnaJ homolog subfamily B member 6 (Heat shock protein J2) (HSJ-2) | 7 | q36.3 | 208811_s_at | |
| | | (MSJ-1) (HHDJ1) (MRJ). [Source: Uniprot/SWISSPROT; Acc: O75190] | | | | |
| BAG4 | 9530 | BAG family molecular chaperone regulator 4 (BCL2-associated athanogene 4) | 8 | p12 | 228189_at | high |
| | | (BAG-4) (Silencer of death domains). [Source: Uniprot/SWISSPROT; | | | | |
| | | Acc: O95429] | | | | |
| STK3 | 6788 | Serine/threonine-protein kinase 3 (EC 2.7.11.1) (STE20-like kinase MST2) | 8 | q22.2 | 204068_at | high |
| | | (MST-2) (Mammalian STE20-like protein kinase 2) (Serine/threonine-protein | | | | |
| | | kinase Krs-1). [Source: Uniprot/SWISSPROT; Acc: Q13188] | | | | |
| RAD21 | 5885 | Double-strand-break repair protein rad21 homolog (hHR21) (Nuclear matrix | 8 | q24.11 | 200608_s_at | high |
| | | protein 1) (NXP-1) (SCC1 homolog). [Source: Uniprot/SWISSPROT; | | | | |
| | | Acc: O60216] | | | | |
| SETX | 23064 | Probable helicase senataxin (EC 3.6.1.—) (SEN1 homolog). | 9 | q34.13 | 201964_at | high |
| | | [Source: Uniprot/SWISSPROT; Acc: Q7Z333] | | | | |
| CDC2 | 983 | Cell division control protein 2 homolog (EC 2.7.11.22) (EC 2.7.11.23) (p34 | 10 | q21.2 | 203213_at | high |
| | | protein kinase) (Cyclin-dependent kinase 1) (CDK1). | | | | |
| | | [Source: Uniprot/SWISSPROT; Acc: P06493] | | | | |
| PTEN | 5728 | Phosphatidylinositol-3,4,5-trisphosphate 3-phosphatase and dual- specificity | 10 | q23.31 | 225363_at | high |
| | | protein phosphatase PTEN (EC 3.1.3.67) (EC 3.1.3.16) (EC 3.1.3.48) | | | | |
| | | (Phosphatase and tensin homolog) (Mutated in multiple advanced cancers 1). | | | | |
| | | [Source: Uniprot/SWISSPROT; Acc: P60484] | | | | |
| PTENP1 | 11191 | Phosphatidylinositol-3,4,5-trisphosphate 3-phosphatase and dual- specificity | 10 | q23.31 | 227469_at | low |
| | | protein phosphatase PTEN (EC 3.1.3.67) (EC 3.1.3.16) (EC 3.1.3.48) | | | | |
| | | (Phosphatase and tensin homolog) (Mutated in multiple advanced cancers 1). | | | | |
| | | [Source: Uniprot/SWISSPROT; Acc: P60484] | | | | |
| TCTN3 | 26123 | Tectonic-3 precursor. [Source: Uniprot/SWISSPROT; Acc: Q6NUS6] | 10 | q24.1 | 212121_at | low |
| HTATIP2 | 10553 | Oxidoreductase HTATIP2 (EC 1.1.1.—) (HIV-1 TAT-interactive protein 2) | 11 | p15.1 | 207180_s_at, | low |
| | | (30 kDa HIV-1 TAT-interacting protein). [Source: Uniprot/SWISSPROT; | | | 209448_at | |
| | | Acc: Q9BUP3] | | | | |
| API5 | 8539 | Apoptosis inhibitor 5 (API-5) (Fibroblast growth factor 2-interacting factor) | 11 | p12 | 201687_s_at | high |
| | | (FIF) (Protein XAGL) (Antiapoptosis clone 11 protein) (AAC- 11). | | | | |
| | | [Source: Uniprot/SWISSPROT; Acc: Q9BZZ5] | | | | |
| BAD | 572 | Bcl2 antagonist of cell death (BAD) (Bcl-2-binding component 6) (Bcl- | 11 | q13.1 | 209364_at | low |
| | | XL/Bcl-2-associated death promoter) (Bcl-2-like 8 protein). | | | | |
| | | [Source: Uniprot/SWISSPROT; Acc: Q92934] | | | | |
| BIRC3 | 330 | Baculoviral IAP repeat-containing protein 3 (Inhibitor of apoptosis protein 1) | 11 | q22.2 | 230499_at | low |
| | | (HIAP1) (HIAP-1) (C-IAP2) (TNFR2-TRAF-signaling complex protein 1) | | | | |
| | | (IAP homolog C) (Apoptosis inhibitor 2) (API2) (RING finger protein 49). | | | | |
| | | [Source: Uniprot/SWISSPROT; Acc: Q13489] | | | | |
| DNM1L | 10059 | Dynamin-1-like protein (EC 3.6.5.5) (Dynamin-like protein) (Dnm1p/Vps1p- | 12 | p11.21 | 203105_s_at | high |
| | | like protein) (DVLP) (Dynamin family member proline-rich carboxyl-terminal | | | | |
| | | domain less) (Dymple) (Dynamin-related protein 1) (Dynamin-like protein 4) | | | | |
| | | (Dynamin-like protein IV) (HdynIV [Source: Uniprot/SWISSPROT; | | | | |
| | | Acc: O00429] | | | | |
| B3GNT4 | 79369 | UDP-GlcNAc: betaGal beta-1,3-N-acetylglucosaminyltransferase 4 | 12 | q24.31 | 219350_s_at | high |
| | | (EC 2.4.1.—) (Beta3Gn-T4) (BGnT-4) (Beta1,3-N- | | | | |
| | | Acetylglucosaminyltransferase-4). [Source: Uniprot/SWISSPROT; | | | | |
| | | Acc: Q9C0J1] | | | | |
| DIABLO | 56616 | Diablo homolog, mitochondrial precursor (Second mitochondria-derived | 12 | q24.31 | 219350_s_at | high |
| | | activator of caspase) (Smac protein) (Direct IAP-binding protein with low pl). | | | | |
| | | [Source: Uniprot/SWISSPROT; Acc: Q9NR281 | | | | |
| RNF34 | 80196 | E3 ubiquitin-protein ligase RNF34 (EC 6.3.2.—) (RING finger protein 34) | 12 | q24.31 | 219035_s_at | high |
| | | (RING finger protein RIFF) (FYVE-RING finger protein Momo) (Human | | | | |
| | | RING finger homologous to inhibitor of apoptosis protein) (hRFI) | | | | |
| | | (Caspases-8 and -10-associated RING finger protein 1 [Source: | | | | |
| | | Uniprot/SWISSPROT; Acc: Q969K3] | | | | |
| TPT1 | 7178 | Translationally-controlled tumor protein (TCTP) (p23) (Histamine- releasing | 13 | q14.13 | 216520_s_at | low |
| | | factor) (HRF) (Fortilin). [Source: Uniprot/SWISSPROT; Acc: P13693] | | | | |
| ITM2B | 9445 | Integral membrane protein 2B (Transmembrane protein BRI) [Contains: | 13 | q14.2 | 217731_s_at, | low |
| | | ABri/ADan amyloid peptide]. [Source: Uniprot/SWISSPROT; Acc: Q9Y287] | | | 217732_s_at | |
| ACIN1 | 22985 | Apoptotic chromatin condensation inducer in the nucleus (Acinus). | 14 | q11.2 | 201715_s_at | low |
| | | [Source: Uniprot/SWISSPROT; Acc: Q9UKV3] | | | | |
| ARF6 | 382 | ADP-ribosylation factor 6. [Source: Uniprot/SWISSPROT; Acc: P62330] | 14 | q21.3 | 224788_at | high |
| GPR65 | 8477 | Psychosine receptor (G-protein coupled receptor 65) (T cell death- associated | 14 | q31.3 | 214467_at | low |
| | | protein 8). [Source: Uniprot/SWISSPROT; Acc: Q8IYL9] | | | | |
| BUB1B | 701 | Mitotic checkpoint serine/threonine-protein kinase BUB1 beta (EC 2.7.11.1) | 15 | q15.1 | 203755_at | high |
| | | (hBUBR1) (MAD3/BUB1-related protein kinase) (Mitotic checkpoint kinase | | | | |
| | | MAD3L) (SSK1). [Source: Uniprot/SWISSPROT; Acc: O60566] | | | | |
| DNAJA3 | 9093 | DnaJ homolog subfamily A member 3, mitochondrial precursor (Tumorous | 16 | p13.3 | 1554078_s_at | high |
| | | imaginal discs protein Tid56 homolog) (DnaJ protein Tid-1) (hTid-1) | | | | |
| | | (Hepatocellular carcinoma-associated antigen 57). [Source: | | | | |
| | | Uniprot/SWISSPROT; Acc: Q96EY1] | | | | |
| CLN3 | 1201 | Battenin (Protein CLN3) (Batten disease protein). | 16 | p11.2 | 209275_s_at | low |
| | | [Source: Uniprot/SWISSPROT; Acc: Q13286] | | | | |
| APPBP1 | 8883 | NEDD8-activating enzyme E1 regulatory subunit (Amyloid protein-binding | 16 | q22.1 | 202268_s_at | high |
| | | protein 1) (Amyloid beta precursor protein-binding protein 1, 59 kDa) | | | | |
| | | (APP-BP1) (Protooncogene protein 1) (HPP1). [Source: | | | | |
| | | Uniprot/SWISSPROT; Acc: Q13564] | | | | |
| P2RX1 | 5023 | P2X purinoceptor 1 (ATP receptor) (P2X1) (Purinergic receptor). | 17 | p13.2 | 210401_at | low |
| | | [Source: Uniprot/SWISSPROT; Acc: P51575] | | | | |
| TNFSF12 | 8742 | Tumor necrosis factor ligand superfamily member 12 (TNF-related weak | 17 | p13.1 | 209500_x_at | low |
| | | inducer of apoptosis) (TWEAK) (APO3 ligand) [Contains: Tumor necrosis | | | | |
| | | factor ligand superfamily member 12, membrane form; Tumor necrosis factor | | | | |
| | | ligand superfamily member 12, secreted for [Source: Uniprot/SWISSPROT; | | | | |
| | | Acc: O43508] | | | | |
| TNFSF13 | 8741 | Tumor necrosis factor ligand superfamily member 12 (TNF-related weak | 17 | p13.1 | 210314_x_at | low |
| | | inducer of apoptosis) (TWEAK) (APO3 ligand) [Contains: Tumor necrosis | | | | |
| | | factor ligand superfamily member 12, membrane form; Tumor necrosis factor | | | | |
| | | ligand superfamily member 12, secreted for [Source: Uniprot/SWISSPROT; | | | | |
| | | Acc: O43508] | | | | |
| TP53 | 7157 | Cellular tumor antigen p53 (Tumor suppressor p53) (Phosphoprotein p53) | 17 | p13.1 | 201746_at | high |
| | | (Antigen NY-CO-13). [Source: Uniprot/SWISSPROT; Acc: P04637] | | | | |
| PSMC5 | 5705 | 26S protease regulatory subunit 8 (Proteasome 26S subunit ATPase 5) | 17 | q23.3 | 209503_s_at | high |
| | | (Proteasome subunit p45) (p45/SUG) (Thyroid hormone receptor- interacting | | | | |
| | | protein 1) (TRIP1). [Source: Uniprot/SWISSPROT; Acc: P62195] | | | | |
| SMARCD2 | 6603 | SWI/SNF-related matrix-associated actin-dependent regulator of chromatin | 17 | q23.3 | 209503_s_at | high |
| | | subfamily D member 2 (60 kDa BRG-1/Brm-associated factor subunit B) | | | | |
| | | (BRG1-associated factor 60B). [Source: Uniprot/SWISSPROT; | | | | |
| | | Acc: Q92925] | | | | |
| BIRC5 | 332 | Baculoviral IAP repeat-containing protein 5 (Apoptosis inhibitor survivin) | 17 | q25.3 | 202095_s_at | high |
| | | (Apoptosis inhibitor 4). [Source: Uniprot/SWISSPROT; Acc: O15392] | | | | |
| SOCS3 | 9021 | Suppressor of cytokine signaling 3 (SOCS-3) (Cytokine-inducible SH2 | 17 | q25.3 | 206360_s_at | low |
| | | protein 3) (CIS-3) (STAT-induced STAT inhibitor 3) (SSI-3). | | | | |
| | | [Source: Uniprot/SWISSPROT; Acc: O14543] | | | | |
| PIK3R2 | 5296 | Phosphatidylinositol 3-kinase regulatory subunit beta (PI3-kinase p85- | 19 | p13.11 | 1568629_s_at | low |
| | | subunit beta) (PtdIns-3-kinase p85-beta). [Source: Uniprot/SWISSPROT; | | | | |
| | | Acc: O00459] | | | | |
| PLEKHF1 | 79156 | Pleckstrin homology domain-containing family F member 1 (PH domain- | 19 | q12 | 219566_at | low |
| | | containing family F member 1) (PH and FYVE domain-containing protein 1) | | | | |
| | | (Phafin-1) (Lysosome-associated apoptosis-inducing protein containing PH | | | | |
| | | and FYVE domains) (Apoptosis-inducing prot [Source: Uniprot/SWISSPROT; | | | | |
| | | Acc: Q96S99] | | | | |
| DEDD2 | 162989 | DNA-binding death effector domain-containing protein 2 (FADD-like anti- | 19 | q13.2 | 225434_at | low |
| | | apoptotic molecule 3) (DED-containing protein FLAME-3). | | | | |
| | | [Source: Uniprot/SWISSPROT; Acc: Q8WXF8] | | | | |
| NUP62 | 23636 | Nuclear pore glycoprotein p62 (62 kDa nucleoporin). | 19 | q13.33 | 202153_s_at | high |
| | | [Source: Uniprot/SWISSPROT; Acc: P37198] | | | | |
| CD40 | 958 | Tumor necrosis factor receptor superfamily member 5 precursor (CD40L | 20 | q13.12 | 35150_at | low |
| | | receptor) (B-cell surface antigen CD40) (CDw40) (Bp50). | | | | |
| | | [Source: Uniprot/SWISSPROT; Acc: P25942] | | | | |
| SERINC3 | 10955 | Serine incorporator 3 (Tumor differentially expressed protein 1). | 20 | q13.12 | 221473_x_at | low |
| | | [Source: Uniprot/SWISSPROT; Acc: Q13530] | | | | |
| CSE1L | 1434 | Exportin-2 (Exp2) (Importin-alpha re-exporter) (Chromosome segregation 1- | 20 | q13.13 | 201111_at, | high, |
| | | like protein) (Cellular apoptosis susceptibility protein). | | | 201112_s_at, | high, |
| | | [Source: Uniprot/SWISSPROT; Acc: P55060] | | | 210766_s_at | high |
| DONSON | 29980 | Protein downstream neighbor of Son (B17). | 21 | q22.11 | 221677_s_at | high |
| | | [Source: Uniprot/SWISSPROT; Acc: Q9NYP3] | | | | |
| SON | 6651 | SON protein (SON3) (Negative regulatory element-binding protein) (NRE- | 21 | q22.11 | 221677_s_at | high |
| | | binding protein) (DBP-5) (Bax antagonist selected in saccharomyces 1) | | | | |
| | | (BASS1). [Source: Uniprot/SWISSPROT; Acc: P18583] | | | | |
| BID | 637 | BH3-interacting domain death agonist (BID) (p22 BID) [Contains: BH3- | 22 | q11.21 | 211725_s_at | low |
| | | interacting domain death agonist p15 (p15 BID); BH3-interacting domain death | | | | |
| | | agonist p13 (p13 BID); BH3-interacting domain death agonist p11 (p11 | | | | |
| | | BID)]. [Source: Uniprot/SWISSPROT; Acc: P55957] | | | | |
| DGCR6 | 8214 | Protein DGCR6 (DiGeorge syndrome critical region 6). | 22 | q11.21 | 208024_s_at | low |
| | | [Source: Uniprot/SWISSPROT; Acc: Q14129] | | | | |
| DGCR6L | 85359 | Protein DGCR6L (DiGeorge syndrome critical region 6-like protein). | 22 | q11.21 | 208024_s_at | low |
| | | [Source: Uniprot/SWISSPROT; Acc: Q9BY27] | | | | |
| PRODH | 5625 | Proline oxidase, mitochondrial precursor (EC 1.5.99.8) (Proline | 22 | q11.21 | 208024_s_at | low |
| | | dehydrogenase) (Proline oxidase 2) (P53-induced gene 6 protein). | | | | |
| | | [Source: Uniprot/SWISSPROT; Acc: O43272] | | | | |
| BIK | 638 | Bcl-2-interacting killer (Apoptosis inducer NBK) (BP4) (BIP1). | 22 | q13.2 | 205780_at | low |
| | | [Source: Uniprot/SWISSPROT; Acc: Q13323] | | | | |
| TSC22D3 | 1831 | TSC22 domain family protein 3 (Glucocorticoid-induced leucine zipper | X | q22.3 | 207001_x_at | low |
| | | protein) (Delta sleep-inducing peptide immunoreactor) (DSIP- | | | | |
| | | immunoreactive peptide) (Protein DIP) (hDIP) (TSC-22-like protein) | | | | |
| | | (TSC-22R). [Source: Uniprot/SWISSPROT; Acc: Q99576] | | | | |
| ARHGEF6 | 9459 | Rho guanine nucleotide exchange factor 6 (Rac/Cdc42 guanine nucleotide | X | q26.3 | 209539_at | low |
| | | exchange factor 6) (PAK-interacting exchange factor alpha) (Alpha-Pix) | | | | |
| | | (COOL-2). [Source: Uniprot/SWISSPROT; Acc: Q15052] | | | | |

TABLE 2

| | | | Myeloma |
|---|---|---|---|
| | Mapping | Expression | IX |
| Characteristic | (n = 84) | (n = 259) | (n = 1966) |
| Age, years | | | |
| Mean | 64.1 | 64.7 | 64.6 |
| SD | 10.1 | 10.0 | 10.2 |
| Serum | | | |
| β2microglobulin, | 6.0 | 5.9 | 6.1 |
| mg/l | | | |
| Mean | 4.1 | 4.3 | 5.9 |
| SD | 61 | 190 | 1789 |
| Total patients | | | |
| Platelets, 10⁹/l | | | |
| Mean | 34.8 | 247.0 | 247.5 |
| SD | 7.3 | 92.0 | 97.3 |
| Total patients | 84 | 258 | 1880 |
| Haemoglobin, g/dl | | | |
| Mean | 10.3 | 10.5 | 10.8 |
| SD | 1.8 | 1.9 | 4.3 |
| Total patients | 84 | 258 | 1880 |
| Serum albumin, g/l | | | |
| Mean | 34.8 | 34.2 | 34.7 |
| SD | 7.3 | 7.3 | 7.0 |
| Total patients | 84 | 257 | 1858 |
| Deletion 13q | | | |
| % | 44.4 | 45.4 | 45.3 |
| No. total patients | 32 | 109 | 473 |
| Total patients | 72 | 240 | 1043 |
| t (4; 14) | | | |
| % | 9.6 | 15.8 | 11.4 |
| No. total patients | 7 | 38 | 120 |
| Total patients | 73 | 240 | 1052 |
| t (11; 14) | | | |
| % | 14.9 | 17.8 | 13.9 |
| No. total patients | 11 | 43 | 146 |
| Total patients | 74 | 241 | 1047 |
| Follow-up, months | | | |
| Mean | 39.1 | 38.6 | 30.3 |
| SD | 13.7 | 12.5 | 13.8 |
| Survival (OS), | | | |
| months | | | |
| Median | 44 | 43 | 46 |
| Range (min-max) | (1-60) | (0-60) | (0-62) |
| ISS, % of patients | | | |
| 1 | 20.7 | 22.3 | 22.0 |
| 2 | 36.2 | 38.5 | 39.4 |
| 3 | 43.1 | 39.1 | 38.7 |
| Chemotherapy, | | | |
| % of patients | | | |
| CVAD | 29.8 | 25.6 | 28.3 |
| CTD | 28.6 | 31.0 | 28.2 |
| MP | 20.2 | 20.2 | 21.6 |
| CTDa | 21.4 | 23.3 | 21.8 |

TABLE 3
Cox regression

| Factor | Z | P-value |
|---|---|---|
| Clinical factors | | |
| Serum albumin | −2.617 | 8.90 × 10⁻³ |
| β-2-microglobulin | 2.2886 | 0.022 |
| Age | 4.6603 | 3.20 × 10⁻⁶ |
| Signature | | |
| 98 gene signature | 2.0043 | 0.0450 |
| Cytogenetic factors | | |
| Deletion 13q | 0.0827 | 0.930 |
| t (4; 14) | −1.4052 | 0.160 |
| t (11; 14) | 1.372 | 0.170 |
| Deletion 17p | −0.687 | 0.490 |
| Deletion 16q | −1.6872 | 0.0920 |

TABLE 4
Cox regression

| Factor | Z | P-value |
|---|---|---|
| Signature | | |
| 6 gene signature | 2.217 | 0.027 |
| Cytogenetic factors | | |
| Deletion 13q | −0.354 | 0.72 |
| t (4; 14) | −0.278 | 0.78 |
| t (11; 14) | 1.291 | 0.2 |
| Deletion 17p | −1.396 | 0.16 |
| Deletion 16q | −0.114 | 0.91 |

### REFERENCES

The following references are all incorporated by reference in their entirety.
1. Carrasco D R, Tonon G, Huang Y, et al. High-resolution genomic profiles define distinct clinico-pathogenetic subgroups of multiple myeloma patients. Cancer Cell 2006; 9(4):313-25.
2. Ross F M, Ibrahim A H, Vilain-Holmes A, et al. Age has a profound effect on the incidence and significance of chromosome abnormalities in myeloma. Leukemia 2005; 19(9):1634-1642.
3. Walker B A, Leone P E, Jenner M W, et al. Integration of global SNP-based mapping and expression arrays reveals key regions, mechanisms and genes important in the pathogenesis of multiple myeloma. Blood 2006; 108(5):1733-1743.
4. Zandecki M, Lai J L, Facon T. Multiple myeloma: almost all patients are cytogenetically abnormal. Br. J. Haematol. 1996; 94(2):217-227.
5. Jenner M W, Leone P E, Walker B A, et al. Gene mapping and expression analysis of 16q loss of heterozygosity identifies WWOX and CYLD as being important in determining clinical outcome in multiple myeloma. Blood 2007; 110(9):3291-300.
6. Walker B A, Morgan G J. Use of single nucleotide polymorphism-based mapping arrays to detect copy number changes and loss of heterozygosity in multiple myeloma. Clin Lymphoma Myeloma 2006; 7(3):186-91.
7. Minna J D, Roth J A, Gazdar A F. Focus on lung cancer. Cancer Cell 2002; 1(1):49-52.
8. Yokota J, Kohno T. Molecular footprints of human lung cancer progression. Cancer Sci 2004; 95(3):197-204.
9. Davies F E, Dring A M, Li C, et al. Insights into the multistep transformation of MGUS to myeloma using microarray expression analysis. Blood 2003; 102(13):4504-4511.
10. Leone P E, Walker B A, Jenner M W, et al. Deletions of CDKN2C in Multiple Myeloma: Biological and Clinical Implications. Clin Cancer Res 2008; 14(19):6033-41.
11. Lin M, Wei L J, Sellers W R, Lieberfarb M, Wong W H, Li C. dChipSNP: significance curve and clustering of SNP-array-based loss-of-heterozygosity data. Bioinformatics. 2004; 20(8):1233-1240.
12. Nannya Y, Sanada M, Nakazaki K, et al. A robust algorithm for copy number detection using high-density oligonucleotide single nucleotide polymorphism genotyping arrays. Cancer Res. 2005; 65(14):6071-6079.
13. Baross A, Delaney A D, Li H I, et al. Assessment of algorithms for high throughput detection of genomic copy number variation in oligonucleotide microarray data. BMC Bioinformatics 2007; 8:368.
14. Gentleman R C, Carey V J, Bates D M, et al. Bioconductor: open software development for computational biology and bioinformatics. Genome Biol 2004; 5(10):R80.
15. Gorringe K L, Jacobs S, Thompson E R, et al. High-resolution single nucleotide polymorphism array analysis of epithelial ovarian cancer reveals numerous microdeletions and amplifications. Clin Cancer Res 2007; 13(16):4731-9.
16. Nagayama K, Kohno T, Sato M, Arai Y, Minna J D, Yokota J. Homozygous deletion scanning of the lung cancer genome at a 100-kb resolution. Genes Chromosomes Cancer 2007.
17. Quesnel B, Preudhomme C, Philippe N, et al. p16 gene homozygous deletions in acute lymphoblastic leukemia. Blood 1995; 85(3):657-63.
18. Stark M, Hayward N. Genome-wide loss of heterozygosity and copy number analysis in melanoma using high-density single-nucleotide polymorphism arrays. Cancer Res 2007; 67(6):2632-42.
19. Chiecchio L, Protheroe R K, Ibrahim A H, et al. Deletion of chromosome 13 detected by conventional cytogenetics is a critical prognostic factor in myeloma. Leukemia 2006.
20. Wuilleme S, Robillard N, Lode L, et al. Ploidy, as detected by fluorescence in situ hybridization, defines different subgroups in multiple myeloma. Leukemia 2005; 19(2):275-8.
21. Rosenwald A, Wright G, Leroy K, et al. Molecular diagnosis of primary mediastinal B cell lymphoma identifies a clinically favorable subgroup of diffuse large B cell lymphoma related to Hodgkin lymphoma. J Exp Med 2003; 198(6):851-62.
22. Aoki-Kinoshita K F, Kanehisa M. Gene annotation and pathway mapping in KEGG. Methods Mol Biol 2007; 396:71-91.
23. Ashburner M, Ball C A, Blake J A, et al. Gene ontology: tool for the unification of biology. The Gene Ontology Consortium. Nat Genet 2000; 25(1):25-9.
24. Sherman B T, Huang da W, Tan Q, et al. DAVID Knowledgebase: a gene-centered database integrating heterogeneous gene annotation resources to facilitate high-throughput gene functional analysis. BMC Bioinformatics 2007; 8:426.

1. A method for determining a prognosis for an individual with multiple myeloma, the method comprising:
determining the expression signature status of cell death genes in a sample obtained from the individual, comprising;
determining the relative expression of each gene in one or more of the following gene pairs:
a) the gene pair in which the first gene is BUB1B and the second gene is HDAC3,
b) the gene pair in which the first gene is CDC2 and the second gene is FIS1,
c) the gene pair in which the first gene is RAD21 and the second gene is ITM2B, and,
using the expression signature status to determine the prognosis for the individual.

2. The method of claim 1, wherein the expression signature status is used to determine the prognosis for the individual by assigning the individual to a high risk group if, for any one of the gene pairs, expression of the first gene is greater than or equal to expression of the second gene.

3. (canceled)

4. The method of claim 2, wherein the individual is assigned to a high risk group if expression of BUB1B is greater than or equal to expression of HDAC3, and optionally
the expression of a cell death gene belonging to the high expressor group is greater than or equal to the expression of a cell death gene belonging to the low expressor group.

5. The method of claim 4, wherein the individual is assigned to a high risk group if expression of BUB1B is greater than or equal to expression of HDAC3, and
a) expression of CDC2 is greater than or equal to expression of FIS1, and/or
b) expression of RAD21 is greater than or equal to expression of ITM2B.

6. The method of claim 2, wherein the individual is assigned to a high risk group if expression of the first gene is greater than expression of the second gene.

7. The method of claim 1, wherein the expression signature status is used to determine the prognosis for the individual by assigning the individual to a high risk group if the expression ratio of the first gene versus the second gene is greater than or equal to 1 for any one of the gene pairs.

8. (canceled)

9. The method of claim 7, wherein the individual is assigned to a high risk group if the expression ratio of BUB1B versus HDAC3 is greater than or equal to 1, and optionally,
the expression ratio of a cell death gene selected from the high expressor group versus a cell death gene selected from the low expressor group is greater than or equal to 1.

10. The method of claim 9, wherein the individual is assigned to a high risk group if the expression ratio of BUB1B versus HDAC3 is greater than or equal to 1, and
a) the expression ratio of CDC2 versus FIS1, and/or
b) the expression ratio of RAD21 versus ITM2B is greater than or equal to 1.

11. The method of claim 1, wherein the relative expression of the first and second genes, or the expression ratio of the first gene versus the second gene, is determined by determining the amounts of mRNA corresponding to each gene in the sample.

12. The method of claim 11, wherein the relative amounts of mRNA corresponding to each gene in the sample are determined.

13. The method of claim 12, wherein the relative amount of mRNA is determined using PCR or real-time quantitative RT-PCR.

14. (canceled)

15. The method of claim 1, wherein the sample obtained from the individual is a tumour sample, a blood sample, a tissue sample or a cell sample.

16. The method of claim 1, comprising:
(a) the step of selecting cells expressing CD138 from the sample obtained from the individual, and determining the expression signature status of cell death genes in the resultant CD138-positive cell enriched sample; and/or
(b) extracting mRNA from a sample obtained from the individual and determining the relative amounts of mRNA corresponding to the first and second genes in the mRNA extract; and/or
(c) the additional step of determining whether the patient has a homozygous deletion in any one of the following cell death genes:
FAF1, CDKN2C, CTSB, TNFRSF10B, TNFRSF10D, BIRC2, BIRC3, ESR1, PLAGL1, SGK, EMP1, FGF14, FOXO1, TFDP1, and KRT18, wherein
the presence of such a homozygous deletion is indicative of a poor prognosis.

17-19. (canceled)

20. The method of claim 1, wherein the prognosis is used for determining clinical treatment given to the individual.

21. The method of claim 20, wherein determining the clinical treatment comprises selecting one or more of the following types of therapy: a type of chemotherapy or a chemotherapy regimen for administration to the individual; a steroid therapy; a thalidomide therapy; a stem cell transplantation therapy; and/or an autograft or allograft therapy.

22. The method of claim 21, wherein the treatment comprises a chemotherapy selected from one or more of: thalidomide-dexamethasone; a bortezomib-based regimen; lenalidomide-dexamethasone; melphalan and prednisone; bortezomib, melphalan and prednisone; lenalidomide plus low-dose dexamethasone; melphalan, prednisone and lenalidomide.

23. The method of claim 1, wherein the method comprises the initial step of obtaining a sample from said individual.

24.-36. (canceled)
